(19) United States
(12) Patent Application Publication
(10) Pub. No.: US 2015/0265628 A1
(43) Pub. Date: Sep. 24, 2015

(54) GENOMIC TESTING FOR EFFECTIVE THERAPIES AND DETERMINATION OF DOSING STRATEGY

(71) Applicant: CompanionDx Reference Lab, LLC, Houston, TX (US)

(72) Inventors: Kevin P. Rosenblatt, Houston, TX (US); Prem P. Gurnani, Houston, TX (US); David W. Lasecki, Cleveland, OH (US)

(21) Appl. No.: 14/664,433

(22) Filed: Mar. 20, 2015

Related U.S. Application Data
(60) Provisional application No. 61/968,572, filed on Mar. 21, 2014.

(51) Int. Cl. A61K 31/5513; G06F 19/00; C12Q 1/68

(52) U.S. Cl. CPC A61K 31/5513; C12Q 1/6883; G06F 19/3456; C12Q 2600/106; C12Q 2600/158

(57) ABSTRACT

Several embodiments disclosed herein relate to methods for calculating a dosing regimen that is tailored specifically to the genetic and metabolic profile of a particular patient. In some embodiments, the dosing regimen is related to administration of the antipsychotic drug clozapine, and the tailoring of the regimen reduces risk of adverse side effects.

### RELATED CASES

This application claims the benefit of U.S. Provisional Application No. 61/968,572, filed Mar. 21, 2014, the entirety of which is incorporated by reference herein.

### BACKGROUND

Patient to patient variability can be a confounding factor in developing a treatment regime. Individual patients react to a given drug, even when doses are the same, in different ways. Development of more tailored approaches to providing a therapy to a particular patient would improve the overall quality of care provided.

### SUMMARY

In many instances, patients do not metabolize drugs in the same way. With the genetic results gained from a blood test or cheek swab in which DNA is collected, doctors can choose the best drug therapy and the correct dosage for those under their care.

With the “trial-and-error” method of prescribing medications, physicians would often have to wait and see whether a patient would respond to a certain medication before judging its efficacy. Pharmacogenomics testing helps physicians and nurses tailor the type and amount of the drug given to a patient, which will increase the efficacy of the medicine while reducing the chance of unwanted side effects or serious drug interactions.

There is a significant gap in the number of patients with schizophrenia (or other associated disorders) who are receiving a treatment that is optimized to their specific indication and/or genetic characteristics. As a result, many patients are failing treatment and/or have to resort to taking numerous drugs to achieve a semblance of normalcy. Widely considered a gold standard therapeutic, clozapine is largely underutilized in the treatment of schizophrenic patients. This is in part because of the potential serious side effects associated with clozapine (e.g., agranulocytosis), but also perhaps due to relatively strict registry requirements for administering the drug and for those patients receiving the drug. However, tailoring a therapy to particular characteristics of a particular patient can reduce the risk and/or severity of side effects. This is particularly advantageous, in several embodiments, because the management/reduction of adverse side effects as a result of clozapine administration allows a very therapeutically efficacious drug to be used in a greater number of patients in need of successful therapy.

Thus, in several embodiments, there are provided methods for treating a schizophrenic patient with an individualized dosing regimen comprising identifying a schizophrenic patient and under consideration for receiving an anti-psychotic medication and ordering a test that includes screening genetic material of patient for one or more markers associated with increased risk of adverse side effects associated with the anti-psychotic medication, if a marker is identified, then determining the expression level (or activity) in the patient of one or more enzymes capable of metabolizing the anti-psychotic medication, categorizing the patient's capacity to metabolize the anti-psychotic medication based on the expression (or activity) level of the one or more enzymes, calculating an individualized dosing regimen for the patient based on the categorization, and treating the patient with the individualized dosing regimen.

Additionally, in several embodiments, there are provided methods for treating a schizophrenic patient with an individualized dosing regimen comprising identifying a schizophrenic patient and under consideration for receiving clozapine and ordering a test that includes screening genetic material of patient for one or more markers associated with increased risk of adverse side effects associated with clozapine, if a marker is identified, then determining the expression level (or activity) in the patient of one or more enzymes capable of metabolizing clozapine, categorizing the patient's capacity to metabolize clozapine based on the expression (or activity) level of the one or more enzymes, calculating an individualized clozapine dosing regimen for the patient based on the categorization, and treating the patient with the individualized clozapine dosing regimen. In several embodiments, the patient may be a patient that is resistant to one or more antipsychotic medications (e.g., attempts to treat the patient's disease have not previously been successful). However, in several embodiments, patients are treated with clozapine as a first line therapy. In some embodiments the identification of markers associated with adverse side effects is a genomic test (e.g., testing the genetic material of the patient). In several embodiments, the test is designed to identify the presence (or absence) of a particular gene, or gene mutation. In several embodiments, the test is designed to identify a particular sequence variant. For example, in several embodiments, the test is designed to identify a sequence variant in at least one gene associated with increased risk of clozapine-induced agranulocytosis. In some embodiments, the sequence variants are present in one or more of the following genes (or variants of these genes) HLA-DQB1, HLA-C, DRD1, NTSR1 and CSF2R.

In several embodiments, the gene associated with increased risk of clozapine-induced agranulocytosis is HLA-DQB1. In several embodiments, the sequence variant the testing is designed to identify is a 6672G>C substitution in HLA-DQB1. Other sequence variants that are associated with clozapine-induced agranulocytosis are tested for in additional embodiments. Also, in additional embodiments, other genes may be screened, for example, those that may be associated with other adverse side effects of clozapine (or another antipsychotic medication).

In several embodiments the screening involves the collection, separation, or isolation of genetic material from a biological sample from the patient. This may include, for example, isolation of DNA, RNA or protein from a sample collected from the patient, such as a blood, saliva (e.g., from a cheek swab), serum, plasma, urine or tissue sample.

In several embodiments, the calculation of the individualized clozapine dosing regimen further comprises an analysis of one or more lifestyle factors (e.g., smoking, alcohol consumption, caffeine intake, diet, etc.) or other diseases that may impact the metabolism of clozapine.

In several embodiments, the one or more enzymes capable of metabolizing clozapine whose expression and/or activity is measured comprises at least one cytochrome p450 oxidase (CYP450). In several embodiments, the CYP450 measure is one or more of CYP450 2D6, CYP450 1A2, and CYP450 3A4. Other CYP450 (and other enzymes) are measured in some embodiments. Depending on the embodiment, the expression, activity, or both are measured. Additional embodiments may also measure binding of the enzyme to the drug, degradation rate of the enzyme, presence or absence of enzyme co-factors, or additional metrics that may impact the ability of a patient to metabolize an anti-psychotic medication, such as clozapine.

In several embodiments, the determination of the expression of the drug-metabolizing enzyme(s) also includes determining the metabolic status or character of enzyme(s) capable of metabolizing the anti-psychotic medication, such as clozapine. In several embodiments, the metabolic status determined for each of the enzymes is that of ultra-rapid metabolizer, extensive metabolizer, intermediate metabolizer, and poor metabolizer. In some embodiments, this categorization is used in conjunction with a weighting factor to determine a single value (or category) that represents the patient's overall ability to metabolize the anti-psychotic medication. In several embodiments, this value is then used to calculate a dosing strategy (e.g., if this patient is an overall poor metabolizer, adjust the dose accordingly—for example, lower the dose, because the patient will take longer to process the drug).

In several embodiments, the method further comprises ordering an analysis of putative drug-drug interactions that may alter the metabolism of the drug being administered, such as clozapine. In several embodiments, the calculating of the individualized clozapine dosing regimen is adjusted to account for any negative drug-drug interactions that increase or decrease the metabolism of clozapine. In embodiments where other anti-psychotic medications are to be administered, the calculating of the individualized dosing regimen is adjusted to account for any negative drug-drug interactions that increase or decrease the metabolism of that particular anti-psychotic medication.

In several embodiments, the screening for markers associated with increased risk for adverse side-effects is performed by next generation sequencing (NGS). In several embodiments, the screening for markers associated with increased risk for adverse side-effects is performed by real-time RT-PCR. In several embodiments, the screening for markers associated with increased risk for adverse side-effects is performed by micro-array analysis. In several embodiments, combinations of these methods are used, depending on the frequency of the marker (e.g., NGS may be performed if looking for a relatively rare marker, with RT-PCR could be used with more frequent markers). In several embodiments, both the screening and the determination of the expression of drug-metabolizing enzymes is performed by one or more of NGS, real-time RT-PCR, micro-array analysis, or other methods (e.g., capillary electrophoresis (CE)-based Sanger sequencing, northern blot analysis, western blot analysis, etc.).

In several embodiments, the method further comprises obtaining at least one blood sample after treating the patient, and evaluating the white blood cell profile of the sample to determine if the patient is developing agranulocytosis. Depending on the embodiment, the method may also include adjusting the individualized clozapine dosing regimen to maintain a clinically acceptable white blood cell profile.

Additionally, there are provided, in several embodiments, methods for calculating an individualized dosing regimen, comprising identifying a treatment-resistant subject requiring anti-psychotic medication, screening the subject for at least one sequence variant in the HLA-DQB1 gene (or other genes); determining the expression and/or activity of one or more genes encoding enzymes responsible for metabolizing the anti-psychotic medication, categorizing the subject's capacity to metabolize the anti-psychotic medication based on the expression and/or activity of each of the enzymes encoded by the one or more genes; and calculating an individualized dosing regimen for the subject based at least in part on the subject's capacity to metabolize the anti-psychotic medication. In several embodiments, the anti-psychotic medication for which the dosing regimen is calculated is clozapine. In several embodiments, the one or more genes encoding enzymes responsible for metabolizing the anti-psychotic medication are members of the cytochrome p450 family. In several embodiments, the genes are one or more of CYP450 1A2, CYP450 2D6, and CYP450 3A4.

As discussed above, in several embodiments the subject's capacity to metabolize the anti-psychotic medication is categorized as one of ultra-rapid metabolizer, extensive metabolizer, intermediate metabolizer, and poor metabolizer and optionally a weighted value is used to determine the to the subject's overall capacity to metabolize the anti-psychotic medication that is used in calculating the individualized dosing regimen. In several embodiments, a starting dose of the anti-psychotic medication determined for the subject is increased, decreased, or unadjusted based on the weighted categorization of the subject's capacity to metabolize the anti-psychotic medication. In some embodiments, the starting dose is increased when the weighted categorization indicates that the subject will metabolize the antipsychotic medication more rapidly, wherein the starting dose is decreased when the weighted categorization indicates that the subject will metabolize the antipsychotic medication more slowly, and wherein the starting dose is unadjusted when the weighted categorization indicates that the subject will metabolize the antipsychotic medication at approximately a normal rate. Further embodiments involve increasing or decreasing the dose of the antipsychotic medication (for example, clozapine) when the subject is also receiving one or more drugs that alters the subject's capacity to metabolize the antipsychotic medication.

In several embodiments, there is also provided a method for treating a schizophrenic patient with an individualized clozapine dosing regimen, comprising identifying a schizophrenic patient resistant to at least one non-clozapine antipsychotic medication and under consideration for receiving clozapine, ordering at least one genomic test, the at least one genomic test comprising (i) screening genetic material isolated from a biological sample collected from the patient for the presence of a 6672G>C substitution in a HLA-DQB1 gene using next generation sequencing (NGS), wherein the substitution is associated with increased risk of clozapine-induced agranulocytosis, (ii) determining, when the NGS screening identifies the substitution, the expression level each of cytochrome p450 oxidase (CYP450) 1A2, CYP450 2D6, and CYP450 3A4; (iii) categorizing the patient's capacity to metabolize clozapine as that of ultra-rapid metabolizer, extensive metabolizer, intermediate metabolizer, or poor metabolizer for each of CYP450 1A2, CYP450 2D6, and CYP450 3A4 based on the determined expression each of CYP450 1A2, CYP450 2D6, and CYP450 3A4; (iv) determining the patient's overall capacity to metabolize clozapine based on the categorization; (v) adjusting a starting dose of clozapine based on the categorization of the determined capacity of the patient to metabolize clozapine, thereby generating an individualized clozapine dosing regimen for the patient; and treating the patient with the individualized clozapine dosing regimen.

In several embodiments, the method further comprises obtaining at least one blood sample after treating the patient, and evaluating the white blood cell profile of the sample to determine if the patient is developing agranulocytosis. Depending on the embodiment, the method may also include adjusting the individualized clozapine dosing regimen to maintain a clinically acceptable white blood cell profile.

There is also provided a method for calculating an individualized clozapine dosing regimen, the method comprising receiving a biological sample collected from a schizophrenic patient, the patient being resistant to at least one non-clozapine antipsychotic medication and under consideration for receiving clozapine; screening genetic material isolated from the biological sample collected from the patient for the presence of a 6672G>C substitution in a HLA-DQB1 gene using next generation sequencing (NGS), wherein the substitution is associated with increased risk of clozapine-induced agranulocytosis; determining, when the NGS screening identifies the substitution, the expression level each of cytochrome p450 oxidase (CYP450) 1A2, CYP450 2D6, and CYP450 3A4; categorizing the patient's capacity to metabolize clozapine as that of ultra-rapid metabolizer, extensive metabolizer, intermediate metabolizer, or poor metabolizer for each of CYP450 1A2, CYP450 2D6, and CYP450 3A4 based on the determined expression each of CYP450 1A2, CYP450 2D6, and CYP450 3A4; determining the patient's overall capacity to metabolize clozapine based on the categorization; obtaining a starting dose of clozapine based on a clinically acceptable use of clozapine; adjusting the starting dose of clozapine based on the categorization of the determined capacity of the patient to metabolize clozapine, thereby calculating an individualized clozapine dosing regimen for the patient. In several embodiments the method also includes reporting the calculated individualized clozapine dosing regimen to a medical care provider for administration to the patient. In several embodiments, the method optionally comprises increasing or decreasing the dose of the clozapine when the patient is also receiving one or more drugs that alters the patient's capacity to metabolize clozapine and/or when the patient has one or more lifestyle characteristics that alters the patient's capacity to metabolize clozapine.

Additional embodiments provide for a method for treating a schizophrenic patient with an individualized clozapine dosing regimen, the method comprising identifying a schizophrenic patient under consideration for receiving clozapine; ordering at least one genomic test, the at least one genomic test comprising; (i) determining, using next generation sequencing (NGS), the expression level each of cytochrome p450 oxidase (CYP450) 1A2, CYP450 2D6, and CYP450 3A4; (ii) categorizing the patient's capacity to metabolize clozapine as that of ultra-rapid metabolizer, extensive metabolizer, intermediate metabolizer, or poor metabolizer for each of CYP450 1A2, CYP450 2D6, and CYP450 3A4 based on the determined expression each of CYP450 1A2, CYP450 2D6, and CYP450 3A4; (iii) determining the patient's overall capacity to metabolize clozapine based on the categorization; (iv) adjusting a starting dose of clozapine based on the categorization of the determined capacity of the patient to metabolize clozapine, thereby generating an individualized clozapine dosing regimen for the patient; and treating the patient with the individualized clozapine dosing regimen. In several embodiments, the method further comprises determining whether any schizophrenic patient is resistant to at least one non-clozapine antipsychotic medication.

In several embodiments, there are provided methods for treating a schizophrenic patient with an individualized clozapine dosing regimen, the method comprising: identifying a schizophrenic patient under consideration for receiving clozapine; ordering at least one genomic test, the at least one genomic test comprising; (i) screening genetic material isolated from a biological sample collected from the patient for the presence of a 6672G>C substitution in a HLA-DQB1 gene using next generation sequencing (NGS), wherein the substitution is associated with increased risk of clozapine-induced agranulocytosis, (ii) determining, when the NGS screening identifies the substitution, the expression level each of cytochrome p450 oxidase (CYP450) 1A2, CYP450 2D6, and CYP450 3A4; (iii) categorizing the patient's capacity to metabolize clozapine as that of ultra-rapid metabolizer, extensive metabolizer, intermediate metabolizer, or poor metabolizer for each of CYP450 1A2, CYP450 2D6, and CYP450 3A4 based on the determined expression each of CYP450 1A2, CYP450 2D6, and CYP450 3A4; (iv) determining the patient's overall capacity to metabolize clozapine based on the categorization; (v) adjusting a starting dose of clozapine based on the categorization of the determined capacity of the patient to metabolize clozapine, thereby generating an individualized clozapine dosing regimen for the patient; and treating the patient with the individualized clozapine dosing regimen.

Some embodiments provide methods for treating a schizophrenic patient with an individualized dosing regimen comprising identifying a schizophrenic patient resistant to at least one antipsychotic medication and under consideration for receiving clozapine; ordering at least one genomic test, the at least one genomic test comprising; (i) screening genetic material of patient for a sequence variant in at least one gene associated with increased risk of clozapine-induced agranulocytosis, wherein the sequence variants are present in a gene selected from the group consisting of HLA-DQB1, HLA-C, DRD1, NTSR1 and CSF2RB; (ii) determining, when the screening identifies at least one sequence variant, the expression level of one or more enzymes capable of metabolizing clozapine; (iii) categorizing the patient's capacity to metabolize clozapine based on the determined expression of the one or more enzymes capable of metabolizing clozapine; and (iv) calculating an individualized clozapine dosing regimen for the patient based on the categorization of the patient's capacity to metabolize clozapine; and treating the patient with the individualized clozapine dosing regimen.

Also provided is a method for treating a schizophrenic patient with an individualized clozapine dosing regimen comprising ordering a test comprising (i) screening genetic material isolated from a biological sample collected from the patient for the presence of a 6672G>C substitution in a HLA-DQB1 gene using next generation sequencing (NGS), wherein the substitution is associated with increased risk of clozapine-induced agranulocytosis, (ii) determining, when the NGS screening identifies the substitution, the expression level each of cytochrome p450 oxidase (CYP450) 1A2, CYP450 2D6, and CYP450 3A4; (iii) categorizing the patient's capacity to metabolize clozapine as that of ultra-rapid metabolizer, extensive metabolizer, intermediate metabolizer, or poor metabolizer for each of CYP450 1A2, CYP450 2D6, and CYP450 3A4 based on the determined expression each of CYP450 1A2, CYP450 2D6, and CYP450 3A4; (iv) determining the patient's overall capacity to metabolize clozapine based on the categorization; (v) adjusting a starting dose of clozapine based on the categorization of the determined capacity of the patient to metabolize clozapine, thereby generating an individualized clozapine dosing regimen for the patient; and treating the patient with the individualized clozapine dosing regimen.

Additional embodiments relate to methods for calculating an individualized clozapine dosing regimen comprising receiving a biological sample collected from a schizophrenic patient under consideration for receiving clozapine; screening genetic material isolated from the biological sample for a sequence variant in at least one gene associated with increased risk of clozapine-induced agranulocytosis; determining, when the screening identifies the sequence variant, the expression level of at least one cytochrome p450 oxidase (CYP450) enzyme; categorizing the patient's capacity to metabolize clozapine as that of ultra-rapid metabolizer, extensive metabolizer, intermediate metabolizer, or poor metabolizer for each CYP450 for which expression is determined; determining the patient's overall capacity to metabolize clozapine based on the categorization; obtaining a starting dose of clozapine based on a clinically acceptable use of clozapine; adjusting the starting dose of clozapine based on the categorization of the determined capacity of the patient to metabolize clozapine, thereby calculating an individualized clozapine dosing regimen for the patient.

Also provided are personalized anti-psychotic medication dosing regimens, the regimens determined by evaluating the risk of a patient for developing one or more adverse side effects from receiving the medication, evaluating the patient's metabolic capacity to process the medication, and calculating an optimal dosing regimen for the patient to minimize adverse side effects while maximizing therapeutic efficacy.

The methods summarized above and set forth in further detail below describe certain actions taken by a practitioner; however, it should be understood that they can also include the instruction of those actions by another party. Thus, actions such as “administering clozapine” include “instructing the administration of clozapine.”

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIGS. 1A-E illustrate a sample series of queries that could be asked of the medical provider when determining in what circumstances the medical provider would prescribe clozapine to a patient.

FIGS. 2A-B illustrate a schematic of various enzymes that metabolize drugs. FIG. 2A shows an example chart of the overall contribution of different types of enzymes to drug metabolism. FIG. 2B shows an example chart of the distribution of drug metabolism across several cytochrome P450 enzymes.

FIG. 3 illustrates examples of categorization of patients based on the degree to which they metabolize a drug.

FIGS. 4A-B illustrates additional characteristics about patients and their metabolism of drugs. FIG. 4A provides characteristics of the way a patient may metabolize an active drug. FIG. 4B provides characteristics of the way a patient may metabolize an inactive prodrug.

FIG. 5 illustrates an example flow-chart of medication management through the use of pharmacogenomics.

FIG. 6 illustrates various examples of the success rate of various drugs on patients.

FIG. 7 illustrates a table of non-limiting examples of pharmacogenomic biomarkers associated with various drugs.

FIG. 8 illustrates an example dosing guideline for the drug Abilify Maintena™.

FIG. 9 illustrates an example flow-chart of a typical medical approach for determining effective therapies and dosing strategies for a patient.

FIG. 10 illustrates various non-limiting examples of CYP enzymes and its metabolism of various antipsychotic medications.

FIGS. 11A-B illustrate various examples of inducers, inhibitors or agents that otherwise can alter the expression and/or activity of various CYP450 enzymes.

FIG. 12 illustrates an example flow-chart of the pharmacogenomic approach for determining effective therapies and dosing strategies for a patient.

FIG. 13 illustrates a schematic with a non-limiting example of a process decision tree that is in accordance with several embodiments disclosed herein.

FIGS. 14A-D illustrate graphs reflecting a survey of 5,311 patients and the spread of patient age, gender and the number of medications each patient is taking. FIGS. 14A-B illustrate the correlation of age and gender with number of medications taken. FIGS. 14C-D illustrate the number of patients and the number of medications taken

FIGS. 15A-I illustrate graphs reflecting the survey of patients from FIGS. 14A-D and the phenotype frequency for the enzymes CYP2D6, CYP2C9 and CYP2C19.

### DETAILED DESCRIPTION

The following discussion is presented to enable a person skilled in the art to make and use one or more of the described embodiments. The general principles described herein may be applied to embodiments and applications other than those detailed below without departing from the spirit and scope of the disclosure. Indeed, the described embodiments are not intended to be limited to the particular embodiments shown, but are to be accorded the widest scope consistent with the principles and features disclosed or suggested herein.

### General

Several embodiments disclosed in the present application relate generally to methods and systems involving development of a patient-specific drug dosing regimen. More specifically, some embodiments of the present application relate generally to methods involving pharmacogenomics testing to calculate or determine an individualized dosing strategy for a subject based, at least in part, on the results obtained during the pharmacogenomics testing.

In some embodiments, a treatment resistant subject or patient (hereinafter “patient”) is identified. The patient can be diagnosed as being one or more of schizophrenic, having a schizoaffective disorder, and/or having schizoaffective disorder and experiencing suicidal ideation. In several embodiments, the patient is resistant to treatment commonly associated with the corresponding diagnosis (e.g., resistant to one or more of drug treatment, psychological therapy, and the like).

In some embodiments, after a treatment resistant patient is identified, a genomic analysis is performed on the patient. Specifically, in several embodiments, pharmacogenomics testing for the Major histocompatibility complex, class II, DQ beta 1, (HLA-DQB1) gene is performed on the patient (or testing on another equivalent gene, gene fragment, or biomarker associated with schizophrenia). Patients who have the HLA-DBQ1 gene are at a significantly higher risk of developing certain side effects as a result of drug administration to treat schizophrenia. In particular, patients who express HLA-DBQ1 are at an increased risk for developing clozapine-induced agranulocytosis when treated with clozapine. In several embodiments, the increased risk can approach 15-20% (e.g., certain patients were calculated to have up to a 16.9 fold increase when the HLA-DBQ1 gene is detected) (see, Athanasiou et al., Candidate Gene Analysis Identifies a Polymorphism in HLA-DQB1 Associated With Clozapine-Induced Agranulocytosis, J. Clin. Psychiatry, Vol. 72(4):458-463 (2011), the entirety of which is incorporated by reference herein. In several embodiments, additional or alternative (e.g., biological equivalent or redundant) genes are also tested, depending on the embodiment, and are also associated with increased risk of agranulocytosis or other adverse side effects. Table 1 depicts various alleles of the HLA-DQB1 that are tested, depending on the embodiment.

TABLE 1
Alleles of HLA-DQB1

| Serotype | DQB1 allele |
|---|---|
| DQ2 | *0201 |
| | *0202 |
| | *0203 |
| DQ4 | *0401 |
| | *0402 |
| DQ5 | *0501 |
| | *0502 |
| | *0503 |
| | *0504 |
| DQ6 | *0601 |
| | *0602 |
| | *0603 |
| | *0604 |
| | *0605 |
| | *0609 |
| DQ7 | *0301 |
| | *0304 |
| DQ8 | *0302 |
| | *0305 |
| DQ9 | *0303 |

Thus, in several embodiments, if the patient tests positive for the HLA-DBQ1 gene (or other gene associated with adverse effects), the patient may no longer considered for treatment with clozapine, but may instead be evaluated for other therapies. If the patient tests negative for HLA-DBQ1, additional analysis and/or testing can be performed, e.g., analysis and/or pharmacogenomics testing of enzymes and/or genes encoding enzymes relating to the metabolism of clozapine, in order to establish an optimized dosing regimen for that particular patient.

In some embodiments, for example, an HLA-DQB1-negative patient is tested for the expression or activity of CYP450 1A2, CYP450 2D6, and/or CYP450 3A4 genes and/or enzymes. These enzymes, among other possible enzymes, are responsible for the metabolism of clozapine (or other antipsychotic drugs). Therefore, the level of activity or expression of these enzymes in the patient is evaluated to determine how the enzymatic profile of the patient will affect the patient's metabolism of clozapine (and thus how it may impact the patient's individual response to a specific dose or dosing strategy of clozapine). In some embodiments, other enzymes can be identified by the testing. In some embodiments, the testing can be pharmacogenomics testing. In some embodiments, the testing can be any other form of testing known in the art to identify the presence of level of specific enzymes and/or genes encoding such enzymes (e.g., library screening, gene chip analysis, protein analysis, enzymatic activity assays, etc.).

In some embodiments, a treatment resistant patient is considered for treatment with clozapine without determining whether the patient is positive for the HLA-DBQ1 gene. In other embodiments, patients who are not treatment resistant (e.g., those who have not failed other treatments and/or those who have not previously been treated) are considered for treatment with clozapine. In several embodiments, the presently disclosed methods allow optimization of dosing tailored to an individual such that clozapine can be considered as a first line therapy.

In some embodiments, the patient's enzyme activity or level of expression of each gene encoding an enzyme can be categorized. In some embodiments, the a patient can be categorized as ultra-rapid metabolizer (“UR”), extensive metabolizer (“E”), intermediate metabolizer (“TM”), or poor metabolizer (“PM”) for each enzyme that is tested. Thus, for example, a patient can be categorized as IM for 1A2, PM for 2D6, and E for 3A4. In some embodiments, these categorizations can then be used to calculate or determine a specific dosing strategy or routine customized to the patient based at least in part on the results of the enzyme testing.

In some embodiments, other factors, including the patient's lifestyle factors and other drug therapies, can also be accounted for in this determination of a dosing routine. Other factors that can be considered in the determination include other enzymes (e.g., other CYP450 enzymes), drug-drug interactions with a focus on inhibitors or inducers of various CYP450 enzymes (e.g., fluvoxamine, paroxetine, dextromethorphan, etc.) or other drugs that may cause agranulocytosis, seizure, or drugs associated with prolonged QT interval (e.g., ziprasidone, iloperidone, pentamidine, methadone, etc.). For example, smoking and/or eating cruciferous vegetables can affect the activity of the CYP450 1A2 enzyme, and thus these factors can be taken into account in the categorization of the patient's 1A2 enzyme and/or in the determination of a dosing routine. As another example, if the patient is undergoing drug therapy, the effect of the drug and possible drug-drug interactions can be taken into account in the categorization of the patient's enzyme and/or the determination of the dosing routine.

In some embodiments, after the patient's enzyme activity has been evaluated, blood (or other biological samples comprising DNA, such as saliva) may be drawn from the patient to further evaluate any potential risks associated with a potential treatment plan determined or calculated based on the above-described testing. For example, the blood can be tested for abnormal blood events. If abnormal blood events or genetic test results are discovered, the recommended therapy can be adjusted or abandoned.

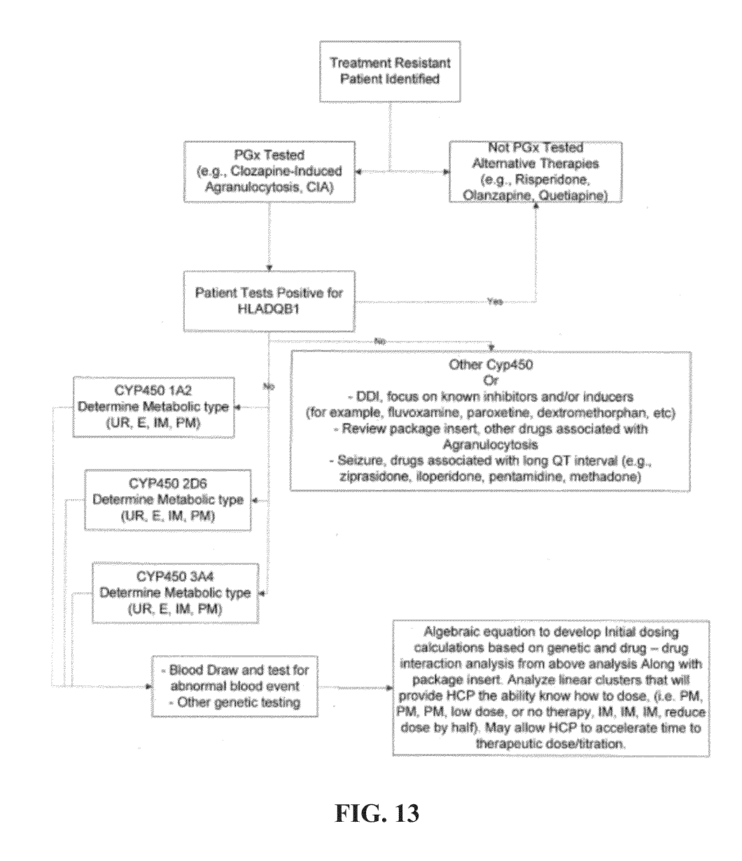

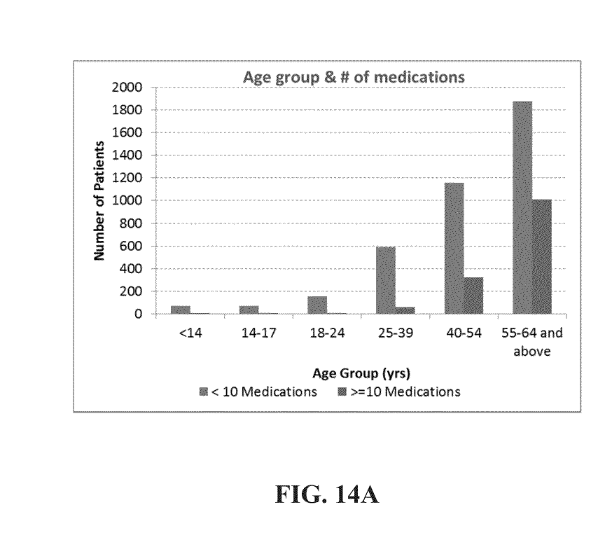

In some embodiments, after the patient's enzyme activity has been analyzed (e.g., tested and categorized), a dosing routine or strategy can be calculated or determined based at least in part on the analysis, e.g., the patient's enzyme levels and/or categorizations as described above. In some embodiments, dosing routines can be calculated using an algebraic equation. These calculations or determinations can also take into account for example, drug-drug interactions, recommendations from the package inserts, etc. In some embodiments, linear clusters can be analyzed to provide healthcare providers the ability to determine a proper individualized dosing strategy. See FIG. 13 as a schematic example of several embodiments disclosed herein. For example, if a patient is categorized as PM (poor metabolizer) for all three enzymes (1A2, 2D6, and 3A4), the patient could be given a low dose or no therapy. As another example, if a patient is categorized as IM (intermediate metabolizer) for all three enzymes (1A2, 2D6, and 3A4), the patient's dose can be reduced by 50% compared to the package insert recommended dosage. Variations in metabolic activity of the various enzymes, in conjunction with consideration of other variables (e.g., lifestyle, diet, other medications, past medical history, potential drug interactions, etc.) are considered in developing specific dosing strategies for individual patients.

In some embodiments, if a patient is HLA-DQB1 positive, the patient may be given alternative treatments or therapies, or may be further tested. Alternative treatments can include risperidone, olanzapine, and/or quetiapine. In some embodiments, the patient can also be tested for enzyme activity (e.g., CYP450 1A2, CYP450 2D6, and/or CYP450 3A4, etc.) as described above, and the results of such tests may be used to determine alternative drug routines.

### Psychotic Disorders

### Overview

Psychosis is a medical condition that, in various forms, impacts a large number of individuals around the world and refers to an abnormal condition of the mind, such as a mental state associated with a loss of distinction between what is an is not reality. People experiencing psychosis may exhibit some personality changes and/or one or more of thought disorders, behavioral disorders, reduced or inappropriate social interaction, or even impairment in carrying out daily life activities.

The term “psychosis” spans relatively normal aberrant experiences to complex and catatonic expressions of schizophrenia and bipolar type 1 disorder. In properly diagnosed psychiatric disorders (e.g., where other causes have been excluded medical and biological laboratory tests), psychosis is a descriptive term for the hallucinations, delusions, sometimes violence, and impaired insight that may occur. As diagnosed psychiatric disorders can thus run a large gamut of signs and symptoms, reflecting some individuality or uniqueness to the disorder, there is a similar need for an ability to tailor a therapy to each individual affected.

In some cases, an excess in dopaminergic signaling may be associated with positive symptoms of psychosis, especially those of schizophrenia. Many currently used antipsychotic medications target the dopamine system; however, no specific mechanism has been identified and variations in drug efficacy indicates that pathophysiology of psychosis is likely much more complex than simply an overactive dopamine system.

### Causes

Many causes of schizophrenia are also causes of psychosis. Historically, organic disorders referred to disorders caused by physical illness affecting the brain (e.g., psychiatric disorders secondary to other conditions) and functional disorders referred to disorders of the functioning of the mind in the absence of physical disorders. However physical abnormalities have been found in illnesses traditionally considered functional, such as schizophrenia. More recently, categorization primarily falls into traditional psychotic illnesses, psychosis due to general medical conditions, and substance-induced psychosis.

Primary psychiatric causes of psychosis may include, but are not limited to, one or more of the following: schizophrenia and schizophreniform disorder; affective (mood) disorders, including severe depression, and severe depression or mania in bipolar disorder (manic depression). People experiencing a psychotic episode in the context of depression may experience self-blaming delusions or hallucinations (or suicidal thoughts), while people experiencing a psychotic episode in the context of mania may form grandiose delusions; schizoaffective disorder, involving symptoms of both schizophrenia and mood disorders; brief psychotic disorder, or acute/transient psychotic disorder; delusional disorder (persistent delusional disorder); or chronic hallucinatory psychosis.

Psychotic symptoms may also be seen in: schizotypal disorder; certain personality disorders, particularly during times of increased stress; major depressive disorder; bipolar disorder in severe mania and/or severe depression; post-traumatic stress disorder; induced delusional disorder; obsessive-compulsive disorder; or dissociative disorders.

In some instances, stress may contribute to and trigger psychotic states. A history of psychologically traumatic events or the recent experience of a stressful event may incite the development of psychosis. Short-lived psychosis triggered by stress is known as brief reactive psychosis, and patients may spontaneously recover. In some cases, individuals may remain in a state of full-blown psychosis for many years, or perhaps have attenuated psychotic symptoms (such as low intensity hallucinations) on an ongoing basis.

Subtypes of psychosis can include, but are not limited to: menstrual psychosis, including circa-menstrual (approximately monthly) periodicity, in rhythm with the menstrual cycle; postpartum psychosis, occurring recently after childbirth; monothematic delusions; myxedematous psychosis; occupational psychosis; stimulant psychosis; tardive psychosis; shared psychosis; or cycloid psychosis

Many medical conditions can cause psychosis, sometimes called secondary psychosis. Examples include, but are not limited to, disorders causing delirium (toxic psychosis); neurodevelopmental disorders and chromosomal abnormalities; neurodegenerative disorders (e.g., Alzheimer's disease, dementia with Lewy bodies, and Parkinson's disease); focal neurological disease (e.g., stroke, brain tumors, multiple sclerosis, epilepsy); malignancy (such as masses in the brain, paraneoplastic syndromes, or drugs used to treat cancer); infectious and postinfectious syndromes (e.g., infections causing delirium, viral encephalitis, HIV, malaria, Lyme disease, syphilis); endocrine disease (e.g., hypothyroidism, hyperthyroidism, adrenal failure, Cushing's syndrome, hypoparathyroidism and hyperparathyroidism); puerperal psychosis; inborn errors of metabolism (e.g., succinic semialdehyde dehydrogenase deficiency, porphyria, metachromatic leukodystrophy); nutritional deficiency (e.g., vitamin deficiency, such as B₁₂ deficiency); acquired metabolic disorders (e.g., electrolyte disturbances such as hypocalcemia, hypernatremia, hyponatremia, hypokalemia, hypomagnesemia, hypermagnesemia, hypercalcemia, hypophosphatemia, hypoglycemia, hypoxia, and failure of the liver or kidneys); autoimmune and related disorders (e.g., systemic lupus erythematosus (lupus, SLE), sarcoidosis, Hashimoto's encephalopathy, and anti-NMDA-receptor encephalitis; poisoning by therapeutic drugs, by recreational drugs, or by any of a range of plants, fungi, metals, organic compounds, or animal toxins; sleep disorders (e.g., narcolepsy; and/or common ailments such as flu or mumps. Other illnesses or situations may also lead to psychosis, depending on the embodiment.

### Anti-Psychotic Treatments

The treatment of psychosis depends on the specific diagnosis (such as schizophrenia, bipolar disorder or substance intoxication). Often, a first-line psychiatric treatment for is antipsychotic medication. When effective, these medications can reduce the positive symptoms of psychosis in about 7 to 14 days. However, as discussed in more detail below, certain patients become refractory to antipsychotic medications, while other patients are nonresponsive to the medication from the outset. In several embodiments, the methods, systems and kits disclosed herein are used to identify a subset of patients within this pool of these nonresponsive patients, develop a tailored therapy for each member of that subset, and/or treat that individual patient with the developed tailored therapy.

The choice of which antipsychotic medication to use is based on the benefits, risks, and costs. For many medical practitioners, the choice is one driven by past experience with a particular medication coupled with an informal assessment of the characteristics of a patient in question as compared to a patient for which treatment was effective. However, this is essentially a “trial and error” approach. It is debatable whether, as a class, typical or atypical antipsychotics are better, though generally, the consensus is that amisulpride, olanzapine, risperidone and clozapine are some of the most effective medications. Unfortunately, consensus does not lead to effective treatment for all patients, thereby reinforcing the need for development of tailored therapies based on the characteristics of an individual patient.

### Typical Drugs

Typical antipsychotics (sometimes referred to as first generation antipsychotics, conventional antipsychotics, classical neuroleptics, traditional antipsychotics, or major tranquilizers) are a class of antipsychotic drugs first developed in the 1950s and used to treat psychosis (in particular, schizophrenia). Typical antipsychotics may also be used for the treatment of acute mania, agitation, and other conditions. The first typical antipsychotics to enter clinical use were the phenothiazines. Second-generation antipsychotics are known as atypical antipsychotics (discussed in more detail below).

Many typical and atypical antipsychotics share a common mechanism of action, e.g., blockade of receptors in the brain's dopamine pathways. However, atypicals antipsychotics, at least in some cases are less likely to cause extrapyramidal motor control deficiencies (e.g., unsteady Parkinson's disease-type movements, body rigidity and involuntary tremors).

Typical medications can be categorized into three levels of potency. Generally, a measure of “chlorpromazine equivalence” can be used as a comparison of the relative effectiveness of antipsychotics. The measure specifies the amount (mass) in milligrams of a given drug that must be administered in order to achieve desired effects equivalent to those of 100 milligrams of chlorpromazine. Agents with a chlorpromazine equivalence ranging from 5 to 10 milligrams would be considered “medium potency”, and agents with 2 milligrams would be considered “high potency”.

Low potency drugs include, but are not limited to, chlorpromazine (largactil, thorazine, or aminazin); chlorprothixene (taractan, tarasan, or truxal); levomepromazine (levinan, levoprome, nozinan, or tisercin); mesoridazine (serentil); periciazine (neulactil, or neuleptil); promazine (propazin); thioridazine (aldazine, mellaril, melleril, ridazine, rideril, sonapax, thiodazine, or thioril).

Medium potency drugs include, but are not limited to, loxapine (loxapac, adasuve, or loxitane); molindone (moban); perphenazine (trilafon, fentazin, or etaperazin); thiothixene (navane, or thixit).

High potency drugs include, but are not limited to, droperidol (dehydrobenzperidol, droleptan, inapsine, or xomolix); flupentixol (depixol, or fluanxol); fluphenazine (anatensol, modecate, permitil, prolixin, or moditen depo); haloperidol (dozic, haldol, serenace, senorm); pimozide (orap); prochlorperazine (buccastem, compro, or stemetil); thioproperazine (majeptil); trifluoperazine (stelazine or triftazin); or zuclopenthixol (clopixol or cisordinol).

### Atypical Drugs

The atypical antipsychotics (known as AAP or as second generation antipsychotics (SGAs)) are a group of antipsychotic drugs, with several atypical antipsychotics having received regulatory approval for schizophrenia, bipolar disorder, autism and as an adjunct in major depressive disorder.

As noted above, both typical and atypical medications tend to block receptors in the brain's dopamine pathways, but atypicals may be less likely to cause extrapyramidal motor control disabilities in patients.

Atypical antipsychotics are often used to treat schizophrenia or bipolar disorder. They are also frequently used for agitation associated with dementia, anxiety disorder, Autism Spectrum Disorder, and off-label uses in the obsessive-compulsive disorder. In dementia, they are generally only being considered after other treatments have failed and if the person in question is at either risk to themselves or others.

Non-limiting examples of atypical antipsychotic drugs include, but are not limited to, amisulpride, aripiprazole, asenapine, blonanserin, clozapine, iloperidone, lurasidone, melperone, olanzapine, paliperidone, quetiapine, risperidone, sertindole, sulpiride, ziprasidone, or zotepine.

### Atypical Drugs—Clozapine

Clozapine is an atypical antipsychotic medication used in the treatment of schizophrenia, and is also sometimes used off-label for the treatment of bipolar disorder and borderline personality disorder. Clozapine is often referred to as the gold standard for the treatment of schizophrenia and is on the World Health Organization's List of Essential Medicine. However, because this drug has numerous severe side effects, clozapine can be considered an underutilized treatment for schizophrenia.

Clozapine is generally used only on patients that have not responded to other anti-psychotic treatments due to a primary side effect of agranulocytosis. Additionally the risk of the development of agranulocytosis often requires ongoing blood tests during treatment, in order to monitor white blood cell levels. This is not only an added expense, but can be an aggravating factor for a patient's schizophrenia/schizoaffective disorder. Thus, in several embodiments clozapine is therefore used, according to several embodiments, for patients that have treatment-resistant schizophrenia/schizoaffective disorder. Treatment-resistant schizophrenia/schizoaffective disorders include individuals experiencing persistent moderate to severe delusions or hallucinations despite two or more clinical trials with antipsychotic drugs. Clozapine may also, according to some embodiments be used for patients with schizophrenia or schizoaffective disorder who are at high risk for suicide. In still additional embodiments, the optimized dosing regimen developed for a particular patient reduces the risk of side effects of clozapine such that clozapine can be used as a first line therapy (e.g., in those patients who have not failed other treatments) and/or irrespective of their status with respect to markers associated with CIA.

Among its efficacy where other treatments have limited success, clozapine may be more effective in reducing symptoms of schizophrenia than older typical antipsychotics. In several embodiments, the patients treated according to the methods disclosed herein have a lower relapse rate, improved patient compliance, and an improvement in symptoms. Advantageously, a tailored dose of clozapine may reduce the propensity for substance abuse in schizophrenic patients.

In 2008, data shows that clozapine was used to treat only 4.4% of patients with schizophrenia in the United States. Veterans Affairs (“VA”) administrative datasets from 2003 to 2007 from 13 VA facilities in the US showed that clozapine utilization in schizophrenia was as low as 0% to 2%. However, outside the US, clozapine use is higher in certain Scandinavian countries and China.

Despite its low usage worldwide, clozapine provides a number of benefits. These benefits include: (1) superiority for positive symptoms in treatment-resistant patients, (2) lower risk for suicide, (3) lower risk for tardive dyskinesia and suppression of established tardive dyskinesia, (4) improvement in cognition contributing to better work and social function, (5) higher quality of life and longer time to discontinuation, and (6) decreased relapse. Despite the positive therapeutic effects, the use of a “gold standard” drug is relatively low. However, in accordance with several embodiments disclosed herein, the development and use of a therapy tailored to a specific patient can improve the effective use of clozapine, by identifying patients suited to receive drug, and optimizing dosing for each patient.

According to several embodiments described herein, clozapine has efficacy in treatment-resistant patients. As discussed above, treatment-resistant schizophrenia patients which are appropriate for consideration whether or not to begin a clozapine trial comprise a subgroup of poor (or functionally impaired) outcome schizophrenia patients which have persistent positive symptoms of at least moderate severity after two or more trials of other antipsychotic drugs, typical or atypical. Approximately 30% of patients with schizophrenia meet these criteria for treatment resistance. Further, in a recent study, the CDC indicated that about 0.75% of the global population is schizophrenic. Therefore, of the 7.1 billion individuals in the world, approximately 52.5 million individuals are affected with schizophrenia, on average. As 30% of patients are treatment resistance, there are potentially 15.8 million individuals who are potentially appropriate for consideration for clozapine treatment. As discussed below, persons with schizophrenia pose a high risk for committing suicide. Statistics show that approximately 1 of 3 individuals with schizophrenia will attempt suicide, and, eventually about 1 out of 10 will succeed in taking their own lives. In some examples, clozapine could reduce the number of suicide without concerns of a serious adverse event.

Common dosages of clozapine in treatment-resistant patients with schizophrenia can range between about 300-600 mg/day. Both lower and higher doses may be sufficient or necessary to achieve efficacy and tolerability. Non-treatment-resistant patients usually respond at doses of 150-400 mg/day. Thus, depending on the embodiment, patients may be dosed with between about 50 and 800 mg/day of clozapine, including about 50 to about 100 mg per day, about 100 to about 150 mg per day, about 150 to about 200 mg per day, about 200 to about 250 mg per day, about 250 to about 300 mg per day, about 300 to about 350 mg per day, about 350 to about 400 mg per day, about 400 mg to about 450 mg per day, about 450 to about 500 mg per day, about 500 to about 550 mg per day, about 550 to about 600 mg per day, about 600 to about 700 mg per day, about 700 to about 800 mg per day, and any dosage in between and including those ranges listed.

Depending on the embodiment, improvement in positive symptoms with clozapine can be realized within a fairly short time. For example, in several embodiments, improvements in schizophrenia symptoms are realized within less than about a month. In some embodiments, positive symptoms are realized within about one to about two months, about two to about three months, about three to about four months, about four to about five months, about five to about six months, and any duration of time between those listed. In still additional embodiments positive symptoms may be realized after about six months of clozapine usage (e.g. 6 to 12 months, or longer). Because of the individual metabolic differences in patients, an initial nonresponsive phase to clozapine treatment does not necessarily preclude a positive overall response after a longer period of administration of clozapine. Advantageously, improvement in measures such as relapse and social function, even after more prolonged treatment with clozapine, may be realized. While a longer duration of clozapine may increase the risk for agranulocytosis (which peaks within the first six months), advantageously, several embodiments of the methods disclosed herein counteract this risk by tailoring the clozapine dosage profile specifically based on, at least in part, the patient's ability to metabolize clozapine. Thus, given the limited treatment options for treatment-resistant patients the choices appear to be (i) patient screening and development of an optimized clozapine administration therapy (which are disclosed herein) or (ii) failing to identify all or nearly all patients who are responders.

Clozapine administration may also reduce suicide risk in patients and lead to an overall positive impact on mortality. Suicide is the major contributor to premature death among patients with schizophrenia. Overall, approximately 30-50% of patients with schizophrenia attempt suicide. While approximately 5% actually die from suicide, this is a fivefold greater rate than the lifetime risk for suicide in the general population of the U.S. Clozapine, in particular when used according to the methods disclosed herein, has the potential to reduce suicidal behavior in patients with schizophrenia and schizoaffective disorder, regardless of treatment-resistance status. Certain post-trial data indicate that, for some patients, the risk of death from suicide while taking clozapine, adjusted for risk factors, was 0.34, compared to 1.0 for perphenazine and 1.58 for quetiapine. It can be estimated from the effects of clozapine on suicide attempts and the proportion of attempters who complete suicide that at least one-third of the approximately 5,000 patients per year with schizophrenia or schizoaffective disorder who commit suicide in the U.S. would not do so had they been treated with clozapine. In fact, given the statistics provided above, the actual number of individuals suffering from schizophrenia who commit suicide may be much higher. The methods disclosed herein would assist in the reduction of attempted suicides, and therefore would likely also reduce successful attempts. Thus, in accordance with several embodiments disclosed herein, there are provided methods to assist in the treatment of survivors of serious previous suicide attempts with clozapine, and initiating clozapine treatment in those patients whose well known other risk factors indicate a high risk for suicide (e.g., hopelessness, substance abuse, family history of suicide and insight).

In several embodiments, clozapine, when administered according to the methods disclosed herein leads to improvement in positive and negative symptoms, general psychopathology, cognition, suicidality/mood, and fewer extrapyramidal side effects (EPS). Additional positive benefits of administration of clozapine according to several embodiments disclosed herein include, but are not limited to, improvement in work and social function, quality of life, lower relapse rate and re-hospitalization. Depending on the individual, certain of these improvements are tied to improvement in cognitive function. As clozapine can improve some domains of cognition in schizophrenia (e.g., verbal fluency, declarative memory, attention, and speeded mental functions), treatment according to the methods disclosed herein cannot only improve cognitive function but also associated positive symptoms. As discussed above, the time for improvement in cognition to become evident and maximal may vary from patient to patient. For example, in several embodiments, improvements in cognition are realized within less than about a month. In some embodiments, positive cognitive benefits are realized within about one to about two months, about two to about three months, about three to about four months, about four to about five months, about five to about six months, and any duration of time between those listed. In still additional embodiments positive symptoms may be realized after about six months of clozapine usage (e.g. 6 to 12 months, or longer). In some embodiments, the dosing schedule for clozapine can be tailored to each patient such that the patient can see benefits much more quickly.

Relapse requiring re-hospitalization is another highly relevant clinical outcome in schizophrenia. In several embodiments, administration of clozapine pursuant to the methods disclosed herein leads to significant improvements in re-hospitalization rates. As such, and because relapse prevention translates into cost effectiveness, reduced burden on relatives, and functional benefits to patients, the presently disclosed systems methods and kits represent an enormous potential savings and improvement in quality of life.

Despite its positive effects, the underutilization of clozapine in the United States has resulted in many missed opportunities for effectively treating the large population of schizophrenia patients in the United States. 3.52 million American adults (or 1.1% of the population that is eighteen years or older) in a given year have schizophrenia. Conservative estimates of the percentages of patients with schizophrenia who are treatment resistant (around 30%) and who have survived a serious suicide attempt (around 10%) suggest that at least 35-40% (as the two groups mentioned partially overlap) should be treated with clozapine. Recent data in the United States (2008) show that clozapine has but a 4.4% market share. According to the 2008 data, of the 1.2 million to 1.4 million American adults who could be effectively treated with clozapine, only about 54,000 to 62,000 are being treated with clozapine. On a global scale, as discussed above, of the 7.1 billion individuals in the world, 0.75% of the global population has schizophrenia. This amounts to roughly 53.25 million people worldwide who suffer from schizophrenia. Using the same statistics provided above, of the 18.64 million to 21.3 million adults worldwide who could be effectively treated with clozapine, only 820,050 to 937,200 are being treated with clozapine. Several embodiments provided herein provide efficient and efficacious methods by which this treatment gap can be reduced and/or eliminated.

### Treatment Failures and Side Effects of Clozapine

As discussed above, one of the major concerns of using clozapine is the development of agranulocytosis in a patient. Other concerns may also contribute to the underutilization of clozapine. These include insulin resistance with increased risk of Type II diabetes, weight gain, and various vascular complications, and possibly myocarditis. However, several embodiments disclosed herein are based on the principle that the doses of clozapine are specifically tailored to an individual's ability to metabolize the drug, thereby maintaining circulating drug levels within the desired therapeutic window and reducing the risk of these adverse side effects

Agranulocytosis, also known as agranulosis or granulopenia, is an acute condition involving a severe and dangerous reduction in white blood cell count (leukopenia). This reduction is most commonly a reduction in the number of circulating neutrophils, which are a major class of infection-fighting white blood cells. People with this condition are at very high risk of serious infections due to their suppressed immune system. Clinically, agranulocytosis presents when the concentration of granulocytes (a major class of white blood cells that includes neutrophils, basophils, and eosinophils) drops below 500 cells/mm³ of blood. A formal diagnosis of agranulocytosis, must also rule out other pathologies with a similar presentation, such as aplastic anemia, paroxysmal nocturnal hemoglobinuria, myelodysplasia and leukemias. This requires a bone marrow examination that shows normocellular (normal amounts and types of cells) blood marrow with underdeveloped promyelocytes. These underdeveloped promyelocytes, if fully matured, would have been the missing granulocytes. Clozapine users in the United States, Canada, and the UK must be nationally registered for monitoring of low WBC and absolute neutrophil counts (ANC).

In addition to clozapine administration, there are other causes of agranulocytosis. For example, many of the following drugs types have been associated with agranulocytosis, including antiepileptics, antithyroid drugs (carbimazole, methimazole, and propylthiouracil), antibiotics (penicillin, chloramphenicol and co-trimoxazole), cytotoxic drugs, gold, NSAIDs (indomethacin, naproxen, phenylbutazone, metamizole), mebendazole, allopurinol, and the antidepressant mirtazapine. Because patients suffering from schizophrenia are often on multiple drugs, certain embodiments of methods disclosed herein take into account other drugs that a patient may be taking that could further increase the risk of clozapine.

As discussed above, it is common for patients who are receiving clozapine to be required to undergo frequent, and often long-term, blood tests to evaluate the granulocyte count, as well as possible withdrawal or dose reduction of the offending agent (e.g., one or more agranulocytosis-inducing medications—with a concurrent risk of relapse of symptoms from, e.g., schizophrenia).

The risk of developing agranulocytosis during clozapine treatment is generally between about 0.7-1.0%. Most cases occur between six weeks and six months of treatment. The chance in the second six months of treatment is 0.70/1,000 patient-years and, after the first year, 0.39/1,000 patient-years.

As a result of the danger of agranulocytosis, patients who are taking clozapine are heavily monitored. Patients are initially monitored weekly for the first six months. If there are no low counts, the patient can be monitored every two weeks for an additional six months. Afterwards, the patient may qualify for every four-week monitoring. However, even this seemingly infrequent monitoring can create a significant challenge for schizophrenic patients. Therefore, advantageously certain embodiments of the treatment methods disclosed herein allow a tailored dose of clozapine that reduce the frequency of follow-up blood counts that are required.

If agranulocytosis should be detected, one option is treatment with granulocyte colony-stimulating factor to restore normal white blood cell levels. Because of the risk of developing infections, the balance of clozapine with blood cell counts is important to the overall well-being of a given patient. In several embodiments, certain patients may be particularly susceptible to development of agranulocytosis, even if they are within the pool of patients that should be able to take clozapine. In some such instances, the methods further comprise administration of one or more types of colony-stimulating factor (e.g., G-CSF, GM-CSF, or combinations thereof).

### Agranulocytosis—Effect on Clozapine Use

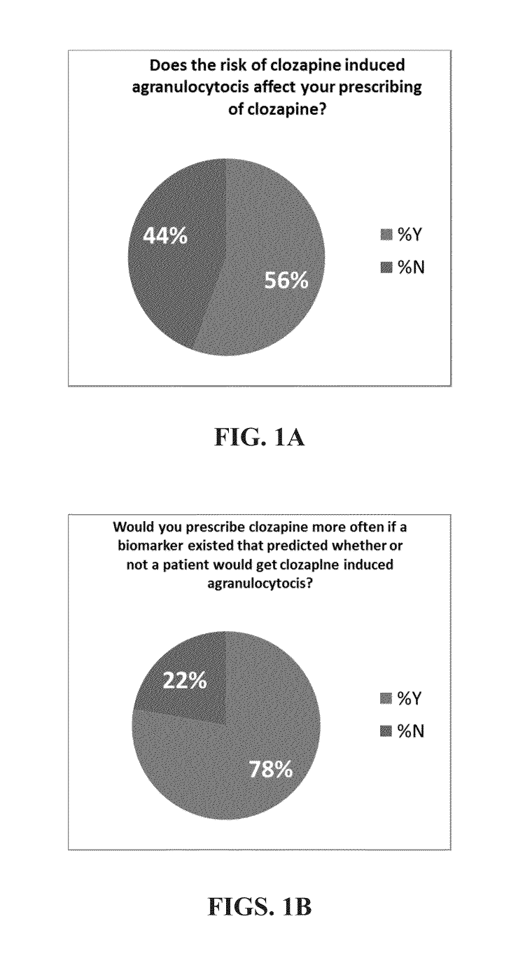

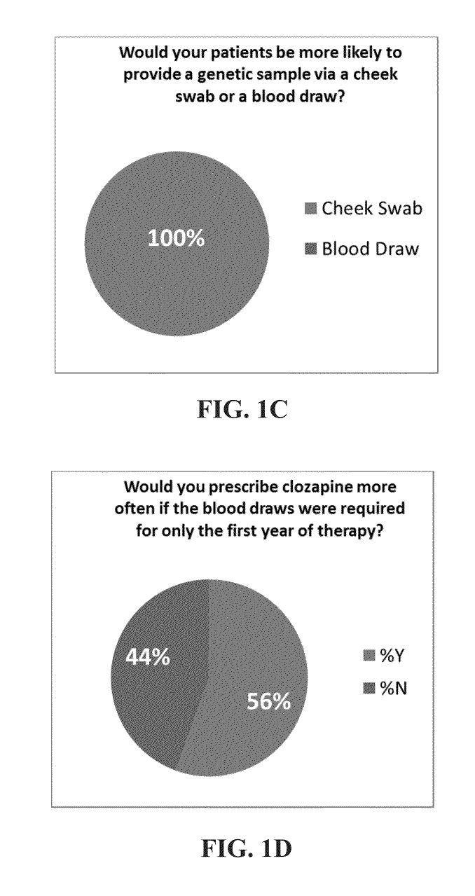

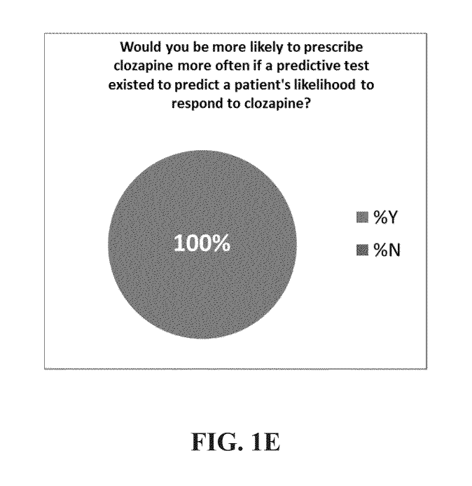

In a companion diagnosis pilot study, a Clozapine Survey Response indicated that the risk of agranulocytosis and the subsequent monitoring of patients for agranulocytosis was a significant hurdle for doctors who considered proscribing clozapine. As seen in FIGS. 1A-E, 56% of the doctors surveyed found that the risk of clozapine induced agranulocytosis affected the doctor's prescribing of clozapine. 78% of doctors surveyed said that they would prescribe clozapine more often if a biomarker existed that predicted whether or not a patient would get clozapine induced agranulocytosis and 100% of doctors surveyed noted that they would be more likely to prescribe clozapine more often if a predictive test existed to predict a patient's likelihood to respond to clozapine.

Also reflected in the survey was the need for a method of treating patients suffering from schizophrenia and related diseases that would eliminate or reduce the number of blood draws. The survey revealed that 100% of doctors noted that patients would be more likely to provide a genetic sample through a cheek swab instead of a blood draw. As well, 56% of doctors noted that they would prescribe clozapine more often if the blood draws were required for only the first year of therapy. The study data reflect the potential for significantly increased efficacious treatment of schizophrenic patients using the screening and personalized dose optimization aspects of the methods disclosed herein.

### Genomic Testing

### Overview

Genomic testing can provide a number of benefits in tailoring a pharmacological regime to a patient's genetic disposition. Genomic testing can frequently be used in a number of ways which include, but are not limited to (1) as a standard of care upon patient admission; (2) as a strategy for disease management; (3) as a tool to potentially reduce polypharmacy (e.g., the use of four or more medications by a patient, generally adults aged over 65 years); (4) as a tool to reduce severe adverse drug reactions; (5) as improving care and quality of life for patients; and (6) as a means to reduce costs associated with ineffective medications.

### Pharmacogenomics—Overview

Pharmacogenomics, generally speaking, is the study of the role of genetics in drug response. Analysis assesses the influence of acquired and inherited genetic variation on drug responses in patients by correlating gene expression or single-nucleotide polymorphisms with one or more of drug absorption, distribution, metabolism and elimination, as well as drug receptor target effects. Pharmacogenomics integrates genomics and epigenetics while also revealing possible effects of multiple genes on drug response.

Pharmacogenomics is used in embodiments of the presently disclosed methods to develop optimized drug therapy, with respect to the patients' genotype, to ensure improved efficacy with reduced adverse effects. The methods disclosed herein seek to free medical providers from a “one-dose-fits-all” approach or trial-and-error method of prescribing. In several embodiments, medical care providers consider their patient's genes, the functionality of the enzymes (or other product) encoded by these genes, and how interplay among these factors may affect the efficacy of the patient's current and/or future treatments. Some embodiments can also provide insight into the reason for the failure of past treatments). Several embodiments reduce poly-pharmacy for certain patients, which further allows for drug and drug combinations to be optimized for each individual's unique genetic makeup, rather than simply adding another drug to a milieu of medications. Whether used to explain a patient's response or lack thereof to a treatment, or act as a predictive tool, several embodiments provided for herein achieve better treatment outcomes, greater efficacy, and reduction of the occurrence of drug toxicities and adverse drug reactions (ADRs). Additionally, in several embodiments, for patients who have lack of therapeutic response to a treatment, alternative therapies can be prescribed that would best suit their requirements.

Genetic variation has been estimated to account for 20-95% of the variation in individual responses to medications. A total of 113 drug labels approved by the US FDA (Food and Drug Administration) include information about variability in patient response secondary to genetic variability, a handful including information about more than one gene. Although the primary focus of pharmacogenomic testing has been on improving drug selection and dosing in patient populations or individuals, a secondary potential benefit of testing may be the improvement of medication adherence. Poor medication adherence is a common problem, particularly in patients with chronic conditions, resulting in greater morbidity, mortality and health-care costs.

In addition, a pseudo-placebo effect may also be at play, as a patient learning that their genetic likelihood of having a positive therapeutic response or not having an adverse effect from the medication may increase the perceived efficacy/necessity or decrease patient concern. Together these can ultimately improve medication adherence, and therapeutic outcome. Actively engaging patients in medication selection or dosing decisions based upon the results of pharmacogenomic testing may, in several embodiments, increase patient knowledge of their disease and treatment options and increase confidence in a physician's recommendations. As well, in some embodiments, increasing patient adherence can potentially reduce the administrative burden on the physician, nursing staff, or the pharmacy dispensing the drug, thereby reducing overall costs to society while improving quality of care. These factors can contribute to reduce patient anxiety and overall costs associated with trial and error of medications for treatment and follow-up care required for management of adverse effects. These reduced burdens can thereby improve long-term outcomes and health costs.

Several different factors may account for the differences in pharmacogenomics characteristics from patient to patient. These can include, but are not limited to, for example: (1) genes affecting the drug's pharmacokinetics, (2) genes affecting the drug's targets and efficacy, and (3) genes predicting the occurrence of disease development, also known as prognostic markers. Thus, according to several embodiments disclosed herein, the methods integrate one or more of these factors to develop a therapy tailored to the genetic makeup of a particular individual.

### Pharmacogenomics—Drug Metabolism

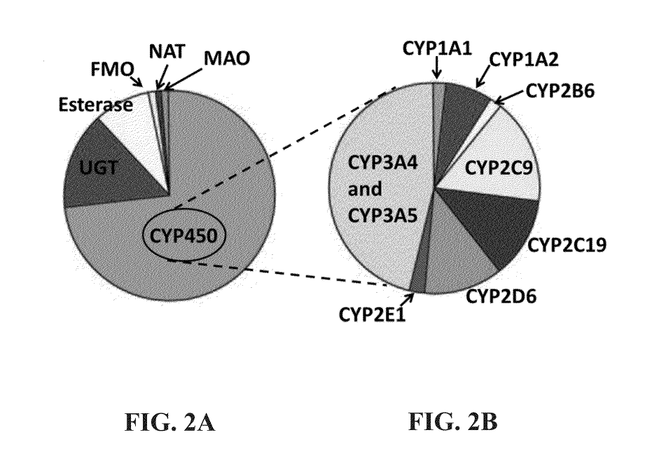

despite the vastly complex network of pathways and medications that are taken, there are 5-7 genes that metabolize more than 90% of all oral medications; 7-12 cover many more and the clearance of IV medications as well. FIGS. 2A-B illustrate the metabolism of individual enzyme systems of marketed drugs in the liver. FIG. 2A illustrates the contribution of individual enzyme systems. As illustrated in FIG. 2A, “UGT” stands for “uridine dinucleotide phosphate (UDP) glucuronosyl transferase”; “FMO” stands for “flavin-containing monooxygenase”; “NAT” stands for “N-acetyltransferase”; “MAO” stands for “monoamine oxidase”; and “CYP450” stands for “Cytochrome P450.” FIG. 2B further delineates the contribution of the CYP450 enzyme system by further illustrating the contribution of individual P450s in the metabolism of drugs.

Generally, the Cytochrome P450 (CYP450) enzymes are the most prevalent drug-metabolizing enzymes. CYP450 enzymes are membrane-bound, heme-containing proteins characterized by 450 nm spectral peak when complexed with carbon monoxide. The human CYP family consists of 57 genes, with 18 families and 44 subfamilies. CYP proteins are arranged into families and subfamilies on the basis of similarities identified between the amino acid sequences. Enzymes sharing 35-40% identity are assigned to the same family by an Arabic numeral, and those sharing 55-70% make up a particular subfamily with a designated letter. For example, CYP2D6 refers to family 2, subfamily D, and gene number 6.

From a clinical perspective, some of the most commonly tested CYPs include: CYP2D6, CYP2C19, CYP2C9, CYP3A4 and CYP3A5. These genes account for the metabolism of approximately 80-90% of currently available prescription drugs. Table 2 below provides a summary for some of the medications that take these pathways.

TABLE 2
Drug Metabolism by Cytochrome P450 Enzymes

| | Fraction | |
|---|---|---|
| | of drug | |
| | metabolism | |
| Enzyme | (%) | Example Drugs |
| CYP2C9 | 10 | Tolbutamide, ibuprofen, mefenamic acid, |
| | | tetrahydrocannabinol, losartan, diclofenac |
| CYP2C19 | 5 | S-mephenytoin, amitriptyline, diazepam, |
| | | omeprazole, proguanil, hexobarbital, |
| | | propranolol, imipramine |
| CYP2D6 | 20-30 | Debrisoquine, metoprolol, sparteine, |
| | | propranolol, encainide, codeine, |
| | | dextromethorphan, clozapine, |
| | | desipramine, haloperidol, |
| | | amitriptyline, imipramine |
| CYP3A4 | 40-45 | Erythromycin, ethinyl estradiol, |
| | | nifedipine, triazolam, cyclosporine, |
| | | amitriptyline, imipramine |
| CYP3A5 | <1 | Erythromycin, ethinyl estradiol, |
| | | nifedipine, triazolam, cyclosporine, |
| | | amitriptyline, aldosterone |

CYP2D6 (debrisoquine hydroxylase) is an extensively studied CYP gene and is highly polymorphic in nature. CYP2D6 has involvement in a high number of medication metabolisms (both as a major and minor pathway). More than 100 CYP2D6 genetic variants have been identified.

CYP2C19 is also extensively characterized, with over 28 genetic variants have been identified for CYP2C19, of which affects the metabolism of several classes of drugs, such as antidepressants and proton pump inhibitors.

CYP2C9 constitutes the majority of the CYP2C subfamily, representing approximately 20% of the overall content of the CYP2C enzymes in the liver. It is involved in the metabolism of approximately 10% of all drugs, which include medications with narrow therapeutic windows such as warfarin and tolbutamide. There are approximately 57 genetic variants associated with CYP2C9.

CYP3A4 and CYP3A5 are members of the CYP3A family, which abundant in the liver. CYP3A4 accounts for 29% of CYP3A enzymatic content in the liver. These enzymes also metabolize between 40-50% of the current prescription drugs, with the CYP3A4 accounting for 40-45% of these medications. CYP3A5 has over 11 genetic variants.

### Pharmacogenomics—CYP450 Test

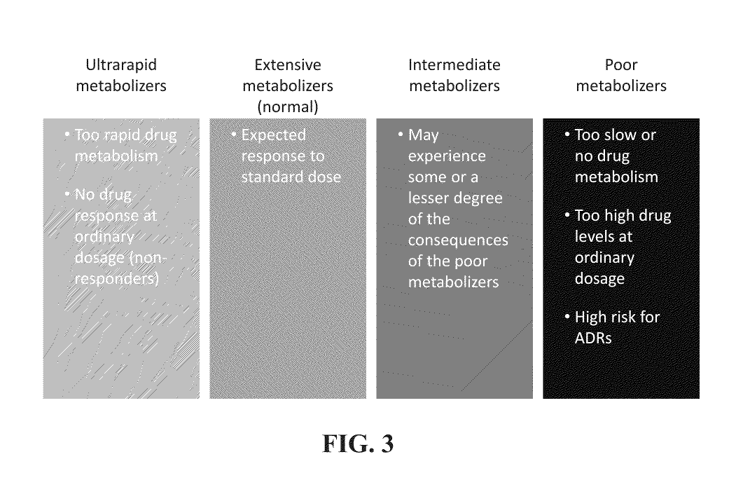

Cytochrome P450 test, according to several embodiments, can be used to categorize a patient into one of the four metabolic profiles or “predicted phenotypes.” FIG. 3 illustrates the four (4) “predicted phenotypes.” Patients that are “ultrarapid metabolizers” have a metabolism that metabolizes the drug very quickly. As a result, that patient has no drug response (or minimal response) at typical dosages. Patients that are “extensive metabolizers” are considered normal as they have an expected response to the standard dose. Patients that are “intermediate metabolizers” may experience some or a lesser degree of the consequences of the poor metabolizers. Finally, the “poor metabolizers” are patients that have slow or no drug metabolism. For these patients, a typical dosage provides elevated drug levels and the patient is therefore at a high risk of adverse drug reactions.

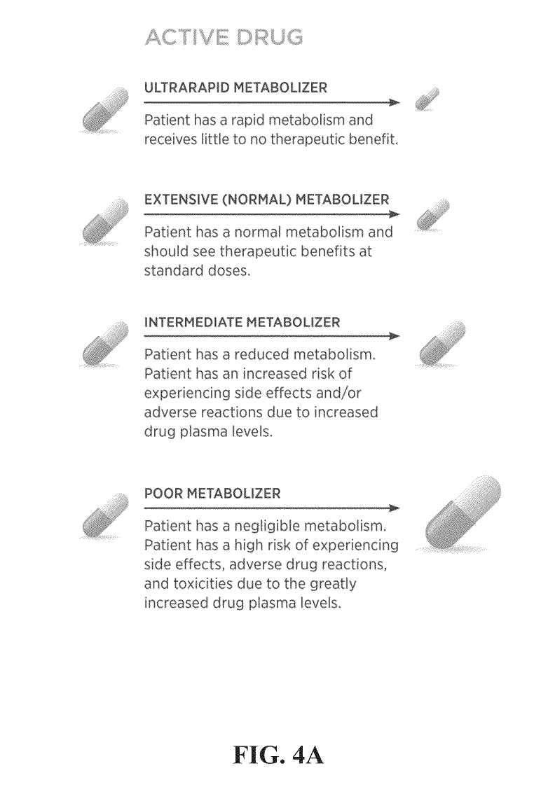

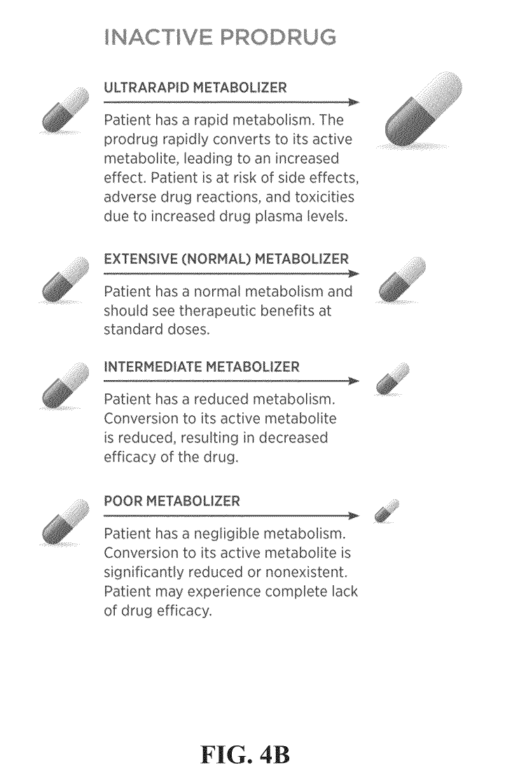

FIGS. 4A-B provide another non-limiting example of CYP 450 testing and categorization of subjects into one of the four “predicted phenotypes” as applied to active drugs and inactive prodrugs. The efficacy of a medication, or a subject's ability to metabolize a medication, is not only based on the above described metabolic categories, but also on the type of drug consumed. Drugs can be classified into two main groups: active drugs and prodrugs. Active drugs refer to drugs that are active upon administration and may be inactivated during metabolism, and prodrugs are inactive until they are metabolized into an active form.

FIG. 4A illustrates the effect of active drugs in patients with varying metabolic capacities. A patient with an “ultrarapid metabolism” receives little to no therapeutic benefit. A patient with an “extensive (normal) metabolism” should see therapeutic benefits at typically employed doses. A patient with an “intermediate metabolism” has an increased risk of experiencing side effects and/or adverse reactions due to increased drug plasma levels. Lastly, a patient with a “poor metabolism” has a high risk of experiencing side effects, adverse drug reactions, and/or toxicities due to the greatly increased drug plasma levels that result from reduced metabolic processing of a drug in question. In several embodiments, the categorization of a patient into one of these categories is associated with application of a “weighting score” or weighting factor”, that can later be used to determine an overall metabolic profile, should multiple CYP450s be assessed (e.g., a score is assigned to a CYP450 for which a patient is an ultrarapid metabolizer and a different score is applied when the patient is a poor metabolizer through another CYP450 pathway).

FIG. 4B illustrates the effect of inactive prodrugs drugs in patients with varying metabolic characteristics. A patient with an “ultrarapid metabolism” rapidly converts the prodrug into its active metabolite, leading to an increased therapeutic effect. The patient is therefore at risk of side effects, adverse drug reactions, and/or toxicities due to increased drug plasma levels. A patient with an “extensive (normal) metabolism” should see therapeutic benefits at typically employed doses. A patient with an “intermediate metabolism” has a reduced rate of converting the active metabolite which results in the decreased efficacy of the drug. Lastly, a patient with a “poor metabolism” converts the inactive prodrug to its active metabolite at a significantly reduced or nonexistent rate. As a result, the patient may experience a significantly reduced of drug efficacy. As discussed above, in several embodiments, the categorization of a patient into one of these categories is associated with application of a “weighting score” or weighting factor”, that can later be used to determine an overall metabolic profile for prodrugs, should multiple CYP450s be assessed (e.g., a score is assigned to a CYP450 for which a patient is an ultrarapid metabolizer and a different score is applied when the patient is a poor metabolizer through another CYP450 pathway).

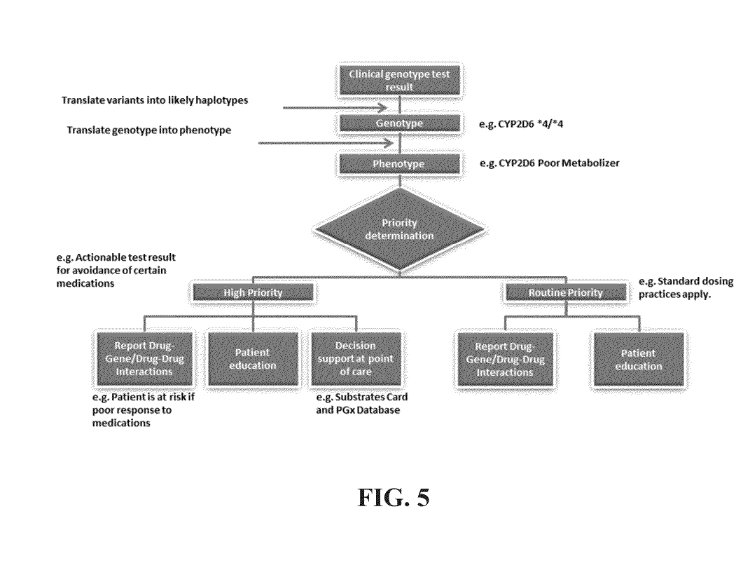

Given the varying metabolic phenotypes that a patient could have, FIG. 5 illustrates a non-limiting example of a medication management process through the use of pharmacogenomics. First, a genotype test is conducted (e.g. a CYP450 pharmacogenomics test) wherein a patient's genetic makeup is determined with respect to the expression level of one or more CYP450 enzymes. In several embodiments, this assessment is preceded by, for example, a determination of the expression levels of HLA-DQB1 and categorization of the patient into a pool of putative clozapine recipients or a pool of alternative therapy recipients. The DNA sequence is translated into likely haplotypes to determine a patient's genotype (e.g. CYP2D6*1/*4). Thereafter, the genotype is used to determine the patient's phenotype (e.g. the patient's metabolism). Once the patient's metabolism is known, a priority determination is made as to whether the patient has a metabolism that would respond to standard dosing practices. If so, in several embodiments, the patient is provided the standard dosing recommended for a given drug (e.g., clozapine). In several embodiments, this standard dose is supplemented with consideration of additional factors such as drug-gene/drug-drug interactions can also be considered and additional patient education.

However, if the patient is found to not respond to standard dosing practices, an actionable test result for the avoidance of certain medications is provided. The test result can provide (1) a report on drug-gene/drug-drug interactions (e.g. patient is at risk if poor response to medication), (2) patient education, or (3) decision support at point of care (e.g. substrates card and pharmacogenomics databases can be used to determine what drug-drug interactions may exist and alternative drugs can be identified).

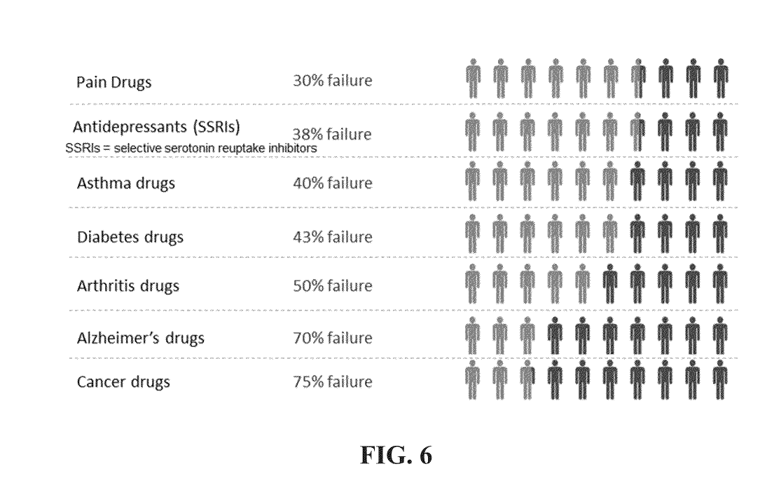

The importance of pharmacogenomics and its use is in conjunction with various embodiments of the methods disclosed herein is described, for example, in the following examples. For example, as illustrated in FIG. 6, patients can respond very differently to the same medicine and drugs, or different dosages of drugs, may not always work the same way for a first versus a second, or subsequent patient. As shown in FIG. 6, 90% of drugs work in 30%-50% of individuals. However, these percentages still lead to roughly $403 billion spent annually on ineffective medicines globally. Advantageously, the methods, systems and kits disclosed herein reduce that financial inefficiency by optimizing therapies on a patient-specific basis.

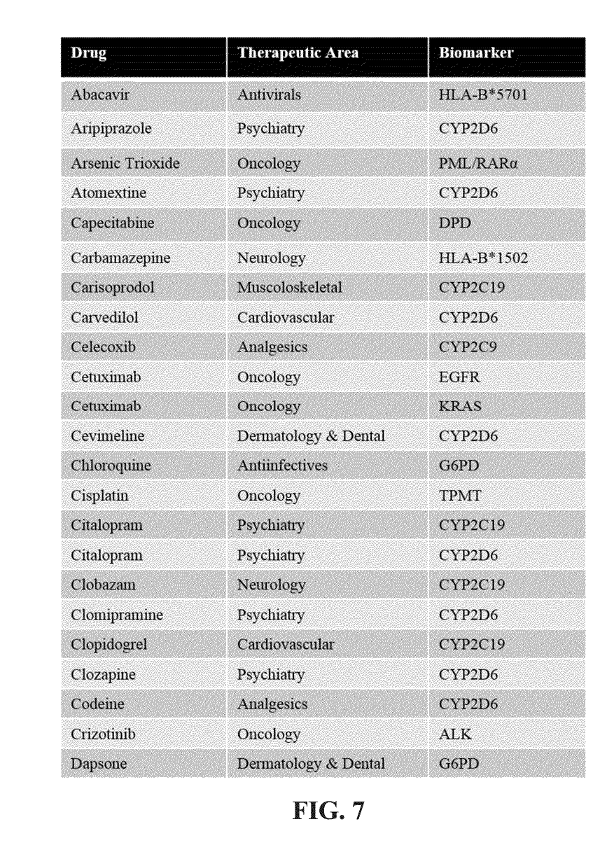

FIG. 7 provides a plurality of non-limiting examples of drugs used in targeted therapeutics and a non-limiting example of an indicative biomarker that can play an important role in identifying responders and non-responders to the indicated medication. As can be appreciated in view of the present disclosure, any of these drugs, or indications, can be processed by the various methodological embodiments disclosed herein to provide a patient-specific therapeutic regime.

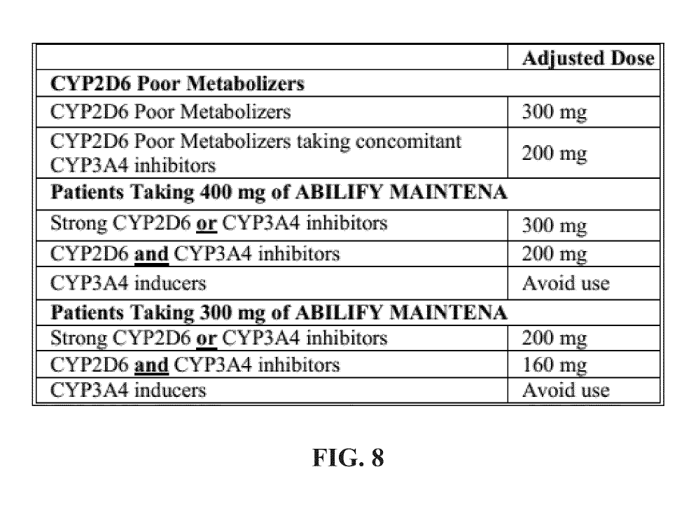

A non-limiting example of how differences in drug metabolism affect the dosage of medication is provided in FIG. 8. FIG. 8 illustrates the product insert for ABILIFY MAINTENA™ (aripiprazole) that provides dosing guidelines. As illustrated, in this example, dosage adjustments are provided for patients who are CYP2D6 poor metabolizers and for patients taking CYP2D6 inhibitors, CYP3A4 inhibitors, or CYP3A4 inducers for more than 14 days. In accordance with several embodiments disclosed herein, a compounded set of determinations (e.g., the metabolic status of a patient across multiple CYP450s) is used to generate an overall dosing regimen for the patient. In several embodiments, putative drug-drug interactions are also accounted for in the determination.

Additional examples of pharmacogenomics in addressing dosing for different metabolizers of other specific drugs for other specific indications are represented, for example in the FDA boxed warning for Plavix (as of Mar. 20, 2010). The label notes that reduced CYP2C19 metabolism in intermediate and poor metabolizers is associated with diminished response to clopidogrel and that pharmacogenetic testing can identify genotypes associated with variability in CYP2C19 activity. However, in contrast to several embodiments of the methods disclosed herein, the information in the new labeling does not directly involve a recommendation for genetic testing prior to administration of the drug.

### Methods of Genetic Testing

Because pharmacogenomic testing is used as the basis, in several embodiments, for determination of a dosing strategy for a particular patient (as compared to another patient) standardized and robust methods for sample procurement and processing are employed in several embodiments.

### Biological Samples

A number of different biological sources can provide for the amounts of genetic material (e.g., DNA or RNA) necessary for genetic testing. Samples can be taken from blood, tumors, serum/plasma, and saliva. Additional sources can be employed, depending on the embodiment, including, for example, tissue samples (e.g., biopsy), blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic, fluid and combinations thereof. In some embodiments, the biological sample is preferably collected from a peripheral location or is naturally excreted or readily excreted by said subject, thereby reducing complications and/or pain associated with repeated testing. The description below provides example methods for attaining DNA, RNA, and/or proteins from a patient's biological sample. The list provided below is not intended to be exclusive and the pharmacogenomic analysis provided in more detail below can include any known methods not provided below.

### RNA-Extraction Methods

The use of salivary diagnostics is beneficial for its noninvasiveness, ease of sampling, and the relatively low risk of contracting infections organisms. Human saliva is useful as an auxiliary biological fluid for disease diagnosis or genetic studies, because the biomolecular composition of saliva changes over time (e.g., in a disease state).

In some embodiments, the high-RNA yield method uses the QIAzol lysis reagent (Qiagen) or similar reagent(s) to isolate RNA from both the cellular pellet and the cell-free salivary supernatant. This allows for a robust, easy, and cost-effective method for isolating high yields of total RNA from saliva for downstream expression studies.

In several embodiments, the methods employ oral whole saliva (˜10 to 200 μL, e.g., ˜10 to ˜50 μL, ˜50 to ˜100 μL, ˜100 to ˜150 μL, ˜150 to ˜200 μL, and any numbers or ranges within those listed) collected from patients. A lysis reagent, such as QIAzol lysis reagent (Qiagen) is used to extract RNA from saliva (both cell-free supernatants and cell pellets), followed by isopropyl alcohol precipitation, cDNA synthesis, and real-time PCR analyses for the genes encoding β-actin (“housekeeping” gene) and various CYP450 enzymes can then be performed. In several embodiments, other methods are used (e.g., high throughput sequencing, gene chip analysis, multiplex PCR, etc.).

In additional embodiments, the method used for RNA-extraction of saliva is through the use of a commercial kit such the Nucleo Spin® RNAII kit (Macherey-Nagel). The method for attaining and isolating the RNA can be through cell-free salivary supernatant or the salivary cellular pellet. One of ordinary skill in the art would readily appreciate the nature of applicable methods to the presently disclosed methods.

In several embodiments, blood is collected for use as a source of genomic material for analysis in a pharmacogenomics analysis. In some embodiments, the blood collected is whole blood. Other embodiments, employ isolated leukocyte preparations. Still additional embodiments employ blood cells separated from plasma.

In several preferred embodiments, the collected whole blood is heparinized upon collection. In several embodiments, the collected whole blood is stored at 4° C. or a lower temperature until a pharmacogenomics analysis is performed.

In several embodiments, a small volume of the previously stimulated blood from each sample is processed to allow determination of the levels of mRNA encoding one or more CYP450 enzymes via a PCR reaction (to amplify corresponding cDNA). After the completion of a PCR reaction, the mRNA (as represented by the amount of PCR-amplified cDNA detected) for one or more CYP450 enzymes is quantified. In certain embodiments, quantification is calculated by comparing the amount of mRNA encoding one or more CYP450 enzymes to a reference value. In several embodiments, the reference value is the expression level of a gene that is not inducible, e.g., a house-keeping gene. In certain such embodiments, beta-actin is used as the reference value. Numerous other house-keeping genes that are well known in the art may also be used as a reference value. In other embodiments, a house keeping gene is used as a correction factor. In still other embodiments, the reference value is zero, such that the quantification of one or more CYP450 enzymes is represented by an absolute number. In several embodiments, expression of CYP450 enzymes from a panel of control individuals (e.g., an average expression in one or more markers measured from a plurality of normal individuals) is used as a baseline comparison for expression.

### Next-Generation Sequencing

Next-Generation Sequencing (NGS) is similar to capillary electrophoresis (CE)-based Sanger sequencing (which is used in several embodiments). In NGS, the bases of a small fragment of DNA are sequentially identified from signals emitted as each fragment is re-synthesized from a DNA template strand. NGS provides the ability for rapid sequencing of large stretches of DNA by extends the sequencing process across many reactions in parallel.

NGS is used in several embodiments as it also provides particularly advantageous experimental design advantages. When, for example, attempting to identify somatic mutations that may only exist within a small proportion of cells in a given sample, NGS allows a region of DNA harboring a mutation to be sequenced at very high levels of coverage, upwards of 1000x. This allows detection of low frequency mutations within the sample population (e.g., when seeking to identify patients with HLA-DQB1 mutations). Also, in some embodiments, genome-wide variant discovery may be employed (e.g., to identify new CYP450 variants or other genes that are mutated and lead to possible sensitivity to clozapine (or other drugs). In these embodiments, NGS can be used to sequence at lower resolution, but process larger sample numbers to achieve greater statistical power within a given population of interest.

NGS is also advantageous in several embodiments because of the ability to quantify RNA activity at much higher resolution than traditional microarray-based methods (though in several embodiments, microarray analysis is used). This sensitivity is important for capturing subtle gene expression changes that may be present in a particular patient (e.g., slight induction of one CYP450 over “normal” ranges). This sensitivity therefore provides a more refined and accurate output of the methods presently disclosed, in other words a more precisely tailored drug administration profile for a particular patient.

NGS sample preparation protocols that are used in several embodiments of the methods disclosed herein are rapid and straightforward and would be readily understood by one of ordinary skill in the art.

### Standard Methodology for Administration of Anti-Psychotics

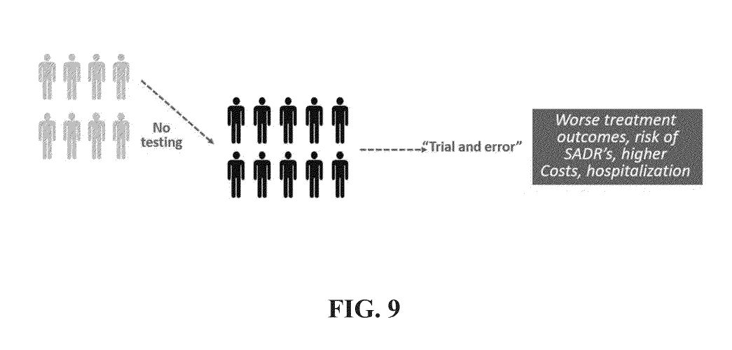

FIG. 9 illustrates an example of the current medical approach to treating schizophrenia patients with clozapine. As no preliminary testing is conducted, a “trial and error” approach is used which can frequently result in compromised treatment outcomes, increased risk of a serious adverse event(s), higher costs, and the risk of hospitalization.

### Patient-Specific Drug Dosing Methodology and Patient-Specific Considerations

The following description deals with the administration of the anti-psychotic clozapine in a patient-specific manner, based at least in part, on a determination of a particular patient's metabolic profile as determined, for example by pharmocogenomic assessment of the patient's CYP450 enzyme expression. However, the general principals from the description below can be applied to a number of other anti-psychotic drugs.

As discussed in detail above, a threshold issue that is addressed by the presently disclosed methods is whether a particular patient is within a pool of patients that should even be considered for receiving clozapine as an antipsychotic medication. In some embodiments, the patient pool is identified by testing for the presence or absence of expression of one or more genes that are associated with sensitivity to clozapine (e.g., mutations that increase the risk of adverse side effects such as agranulocytosis, or other adverse effects discussed herein). In some embodiments, the presence or absence of multiple mutations is assessed and the various mutations are weighted in order to make a determination as to whether the particular patient in question should be considered for receiving clozapine.

The use of pharmacogenomics can take the concerns related to clozapine administration into account, and in accordance with several embodiments disclosed herein, tailor a dosing regimen to a specific patient's capacity for metabolizing clozapine. Drug labels may contain information on genomic biomarkers and can describe drug exposure and clinical response variability; risk for adverse events; genotype-specific dosing; mechanisms of drug action; and polymorphic drug targets and disposition genes. However, the assessment of CYP450 expression and or activity take these general recommendations that are provided on the drug label and transform them into a precise and actionable drug administration regime.

Depending on the embodiment, various markers for risk of adverse effects due to clozapine administration are assessed. In some embodiments sequence variants in one or more of HLA-DQB1, HLA-C, DRD1, NTSR1 and CSF2RB are identified. In some embodiments, presence of specific sequence variants in one or more of these markers is a determining factor in whether a particular patient is at an elevated risk for development of clozapine-induced adverse effects. As discussed above, the HLA locus has also been implicated in the principal dangerous side effect of clozapine, agranulocytosis, which limits the use of this important and effective medication. A sequence variant (6672G>C) in HLA-DQB1 is associated with increased risk for clozapine induced agranulocytosis (“CIA”). The odds of developing CIA are ˜15-20 (16.9 according to one study) times greater in patients who carry this marker compared to those who do not. This marker identifies a subset of patients with an exceptionally high risk of CIA, 1,175% higher than the overall clozapine-treated population under the current blood-monitoring system. Thus, in several embodiments identification of the 6672G>C sequence variant in HLA-DQB1 is used as a threshold for determining whether a patient should be eligible for receiving clozapine as a therapeutic. Once a patient has crossed this threshold, additional pharmacodynamic testing is performed, according to some embodiments, in order to develop a clozapine administration dosing regime that is tailored specifically to that patient's ability to metabolize clozapine, based at least in part on expression of one or more CYP450 enzymes.

### Considerations for Administering Clozapine

To optimize the benefits of clozapine, a number of considerations must be taken into account. These can include, for example, metabolism rate, drug-to-drug interactions, and other lifestyle issues (e.g. diet, caffeine intake, and/or smoking) that can affect the effectiveness of clozapine. As discussed herein, several embodiments of the methods, systems and kits provided address one or more of these factors in the development of a patient-specific drug therapy regimen. Additional information regarding systems for detecting specific variants in genes, determining pharmacogenomics profiles of a patient, and/or monitoring the level of a therapeutic administered can be found in U.S. patent application Ser. No. 14/553,750 and International Application No. PCT/US2014/067446, the entirety of each of which is incorporated by reference herein.

Drug-to-Drug Interactions

It is generally appreciated that initial titration, dosage and adequate duration of treatment, and avoidance of additional add-on antipsychotic drugs until monotherapy with clozapine has been evaluated are important general principles. Assuming a normal metabolic capacity of clozapine, achieving target plasma levels of clozapine (for example greater than or equal to approximately 350 ng/ml), may require periodic measurement at the early phase of treatment and utilization of initial doses up to 900 mg/day. This “loading period” may enhance the benefit-to-risk ratio. In several embodiments, if a patient is on another antipsychotic drug when clozapine is initiated, the other medication is discontinued shortly after adequate dosage of clozapine has been achieved.

In several embodiments, patients may be administered a second antipsychotic drug that acts in concert with clozapine to produce synergistic results. There is controversy about the value of adding risperidone. Some selective serotonin reuptake inhibitors, including paroxetine and fluoxetine can impair the metabolism of clozapine (thereby requiring a reduction in the dose of clozapine). Sertraline and citalopram are not interfering with the metabolism of clozapine. Valproic acid may also increase plasma levels of clozapine, warranting reducing the dosage, in several embodiments. Thus, in several embodiments, concurrent medication is taken into account as drug-to-drug interactions may exist that either can inhibit the effects of clozapine, and thereby require a dosage adjustment.

In some embodiments, because clozapine is a substrate for many cytochrome P450 isozymes (in particular CYP1A2, CYP3A4, and CYP2D6), patients may need to use caution when administering clozapine concomitantly with drugs that are inducers or inhibitors of those enzymes.

In some examples, concomitant use with CYP1A2 inhibitors can increase plasma levels of clozapine, potentially resulting in adverse reactions. In some embodiments, the patient may be required to reduce the clozapine dose (e.g., reduced by about 10%, by about 20%, by about 30%, by about 40%, by about 50%, or more) when co-administered with strong CYP1A2 inhibitors (e.g., fluvoxamine, ciprofloxacin, or enoxacin). Conversely, in some embodiments, the clozapine dose should be increased to the original dose when co-administration of strong CYP1A2 inhibitors is discontinued. In some embodiments, moderate or weak CYP1A2 inhibitors include, but are not limited to, oral contraceptives and caffeine.

In some variants, concomitant treatment with CYP2D6 or CYP3A4 inhibitors (e.g., cimetidine, escitalopram, erythromycin, paroxetine, bupropion, fluoxetine, quinidine, duloxetine, terbinafine, or sertraline) can increase clozapine levels and lead to adverse reactions.

In other embodiments, concomitant treatment with drugs that induce CYP1A2 or CYP3A4 can decrease the plasma concentration of clozapine, resulting in decreased effectiveness. In some embodiments, tobacco smoke is a moderate inducer of CYP1A2. In some embodiments, strong CYP3A4 inducers include carbamazepine, phenytoin, St. John's wort, and rifampin. In such instances, it may be necessary to increase the clozapine dose if used concomitantly with inducers of these enzymes. In further examples, concomitant use of clozapine and strong CYP3A4 inducers are not recommended. In some examples, the clozapine dosage can be reduced when discontinuing co-administered enzyme inducers; because discontinuation of inducers can result in increased clozapine plasma levels and an increased risk of adverse reactions.

In some examples, concomitant treatment with medications that prolong the QT interval or inhibit the metabolism of clozapine can be problematic. Drugs that cause QT prolongation include: specific antipsychotics (e.g., ziprasidone, iloperidone, chlorpromazine, thioridazine, mesoridazine, droperidol, and pimozide), specific antibiotics (e.g., erythromycin, gatifloxacin, moxifloxacin, sparfloxacin), Class 1A antiarrhythmics (e.g., quinidine, procainamide) or Class III antiarrhythmics (e.g., amiodarone, sotalol), and others (e.g., pentamidine, levomethadyl acetate, methadone, halofantrine, mefloquine, dolasetron mesylate, probucol or tacrolimus).

In some embodiments, concomitant use of clozapine with other drugs metabolized by CYP2D6 can increase levels of these CYP2D6 substrates. In some examples, it may be necessary to use lower doses of such drugs than usually prescribed. In some examples, such drugs can include specific antidepressants, phenothiazines, carbamazepine, and Type 1C antiarrhythmics (e.g., propafenone, flecainide, and encainide).

Lifestyle Considerations

Additional factors are also considered, in several embodiments. For example, smoking leads to induction of cytochrome P450 CYP1A2 which will lead to lower plasma clozapine levels. Conversely, smoking cessation may markedly elevate clozapine plasma levels. Depending on the context, plasma levels of clozapine may need to be monitored for at least 3-6 months after smoking cessation and dosage adjustments made as needed. In several embodiments, patients that do receive clozapine are given psychosocial treatment to maximize the benefits from the improvement in psychosis, negative symptoms and cognitive impairment which may emerge in the months after starting a clozapine dosing regimen. Family and group therapy, cognitive behavioral treatment, supportive employment, and activity therapy have may also be helpful, in several embodiments.

### Additional Considerations

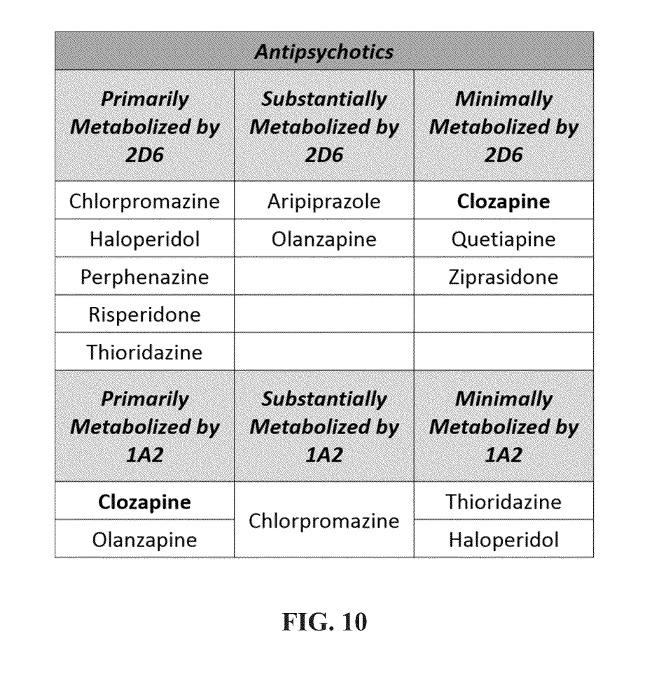

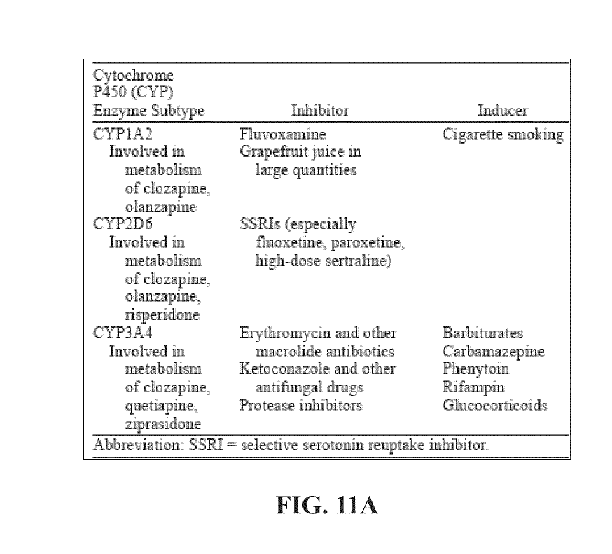

FIG. 10 illustrates certain various antipsychotics and the CYP-mediated clozapine interactions. As illustrated in FIG. 10, CYP1A2 has a major role in the oxidative metabolism of clozapine, with a minor contribution from CYP3A4, and possibly CYP2D6, CYP2C9, and CYP2C19. Interactions mediated by potent CYP1A2 inhibitors (such as fluvoxamine) or inducers (like cigarette smoke) appear to be consistent, predictable and usually clinically significant. As per the Clozaril (clozapine) Package Insert (Novartis, October 2011) stated, “[p]atients taking benzodiazepines, antihypertensives, citalopram, caffeine, tobacco smoke, and inhibitors or inducers of the cytochrome P450 1A2, 2D6, and 3A4 isozyme systems, should be carefully monitored upon clozapine initiation and during therapy. However, in contrast to various embodiments of the methods disclosed herein, the package insert suggests a reactionary monitoring, rather than a proactive testing and dosage regime development.

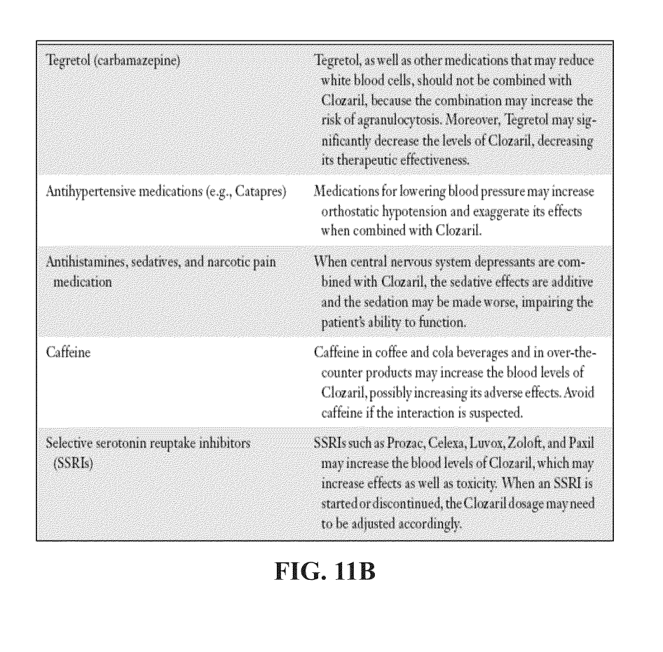

FIG. 11A illustrate various inhibitors and inducers for CYP450 Enzymes. CYP1A2 is inhibited by Fluvoxamine and grapefruit juice in large quantities and is induced by cigarette smoke. CYP2D6 is inhibited by SSRIs (“selective serotonin reuptake inhibitor”) especially fluoxetine, paroxetine, and high-dose sertraline. CYP3A4 is inhibited by erythromycin and other macrolide antibiotics, Ketoconazole and other antifungal drugs, and protease inhibitors. CYP3A4 is induced by barbiturates, carbamazepine, phenytoin, rifampin, and glucocorticoids.

FIG. 11B illustrate various drug-drug interactions. For example, Targretol (carbamazepine), as well as other medications that may reduce white blood cells, should not be combined with clozapine, because the combination may increase the risk of agranulocytosis. Moreover, Tegretol may significantly decrease the level of clozapine, decreasing its therapeutic effectiveness. Antihypertensive medications (e.g. catapres) that are used for lowering blood pressures may increase orthostatic hypotension and exaggerate its effects when combined with clozapine. Antihistamines, sedatives, and narcotic pain medication can serve as central nervous system depressants and, when combined with clozapine, the sedative effects are additive and the sedation may be made worse, impairing the patient's ability to function. Caffeine in coffee and cola beverages and in over-the-counter products may increase the blood levels of clozapine, possibly increasing its adverse effects. Caffeine should be avoided if the interaction is suspected. SSRIs, such as Prozac, Celexa, Luvox, Zoloft, and Paxil may increase the blood levels of clozapine, which may increase effects as well as toxicity. When an SSRI is started or discontinued, the clozapine dosage may need to be adjusted accordingly.

### Pharmacogenomic Approach

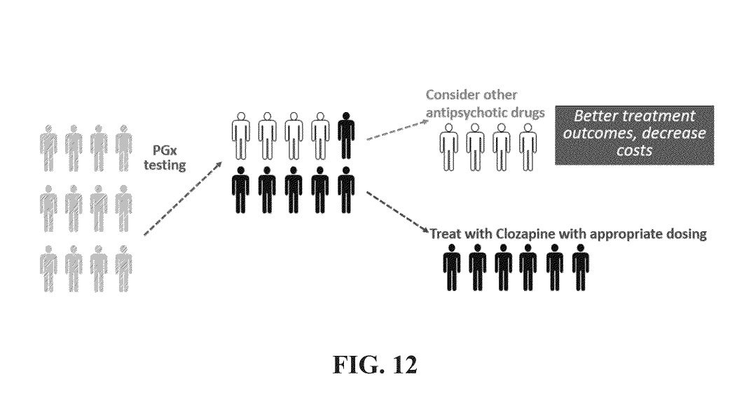

FIG. 12 illustrates an overview of the flowchart for pharmacogenomics testing. As is demonstrated, the considerations discussed above are analyzed for each patient such that each patient is determined to be a candidate for clozapine or other antipsychotic drugs.

FIG. 13 illustrates a detailed flow chart for pharmacogenomics testing in determining whether clozapine is a treatment option for the patient. First, a treatment resistant patient is identified. If the patient is not pharmacogenomics tested, he or she will frequently undergo alternative therapies such as Resperidone, Olanzapine, or Quetiapine. If pharmacogenomics testing is performed, the patient will be tested for clozapine-induced agranulocytosis (“CIA”). As discussed above, the HLA-DQB1 gene can be indicative for the increased risk for CIA. If the patient tests positive for HLADQB1, the patient, in several embodiments, will be directed towards alternative theories (as discussed above) such as Risperidone, Olanzpine, and Quetiapine.

If the patient does not test positive for HLADQB1, the patient is tested for the factors above. These can include enzymes that effect drug metabolism, drug-drug interactions, or other drug inhibitors/inducers. In some examples, the pharmacogenomic test determines the metabolic type for CYP450 1A2 (e.g. UR, E, IM, or PM). In some examples, the pharmacogenomic test determines the metabolic type for CYP450 2D6 (e.g. UR, E, IM, or PM). In some examples, the pharmacogenomic test determines the metabolic type for CYP450 1A2 (e.g. UR, E, IM, or PM). In some examples, the pharmacogenomic test considers other CYP450 enzymes.

In other examples, the pharmacogenomics test considers drug-drug-interactions, with a focus on inhibitors and/or inducers (e.g. fluvoxamine, paroxetine, dextromethorphan). In some examples, the pharmacogenomics test considers the package insert of other drugs the patient is using. In some examples, the pharmacogenomics test considers other drugs associated with agranulocytosis. In some examples, the pharmacogenomics test considers drugs that can induce seizure or drugs associated with long QT intervals (e.g. ziprasidone, iloperidone, pentamidine, methadone).

In some embodiments, once this is accomplished, the patient's blood can be drawn and tested for abnormal blood events. In some embodiments, other genetic testing can be conducted.

In some embodiments, an algebraic equation can be developed to determine initial dosing calculations based on the genetic and drug-drug interaction analysis provided above. The linear clusters of the algebraic equation can be determined that will provide the healthcare professional the ability to know how to dose (i.e. PM, PM, PM, low dose, or no therapy, IM, IM, IM, reduce dose by half). This may allow the healthcare professional to accelerate time to therapeutic dose/titration.

### Additional Advantages of Pharmacogenomics Clozapine Sensitivity Test

In addition to the ability to customize drug dosages to each patient's metabolism and daily routine, a pharmacogenomics clozapine sensitivity test provides for many additional advantages as well. The use of the pharmacogenomics clozapine sensitivity test can help to increase the clinician's ability to provide effective and targeted treatment options to its current consumers diagnosed with treatment resistant schizophrenia/schizoaffective disorder and suicide ideation. As well, the pharmacogenomics clozapine sensitivity test can result in more effective management of resources allocated to treatment resistant individuals. In some examples, this can provide for decreased overall hospitalizations/emergency room visits for the treatment of resistant population and can also increase the overall quality of life for the treatment resistant population.

### EXAMPLES

### Example 1

### Diagnosis Reference Lab Field Experience (“FLEX”) Study

The objective of this study was to assess the ability and applicability of using genomic testing to direct changes in treatment and dosing for patients receiving clozapine. The study was a cross-sectional study design with collection on data from 5311 patient.

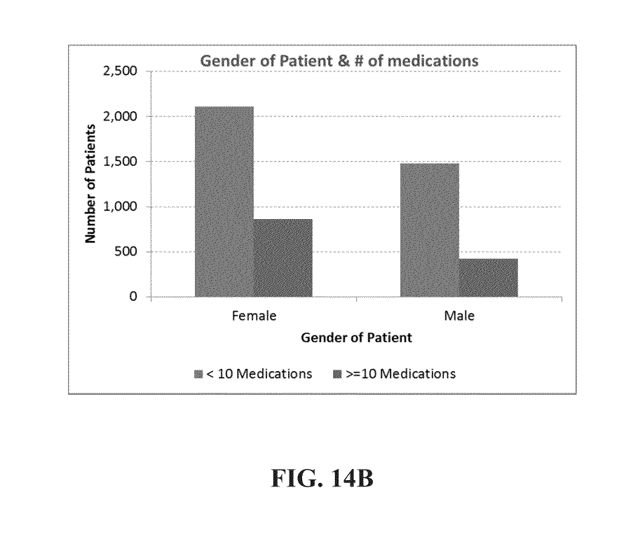

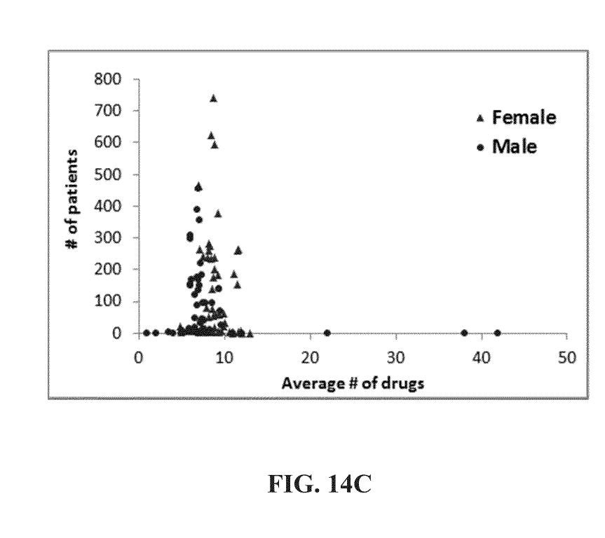

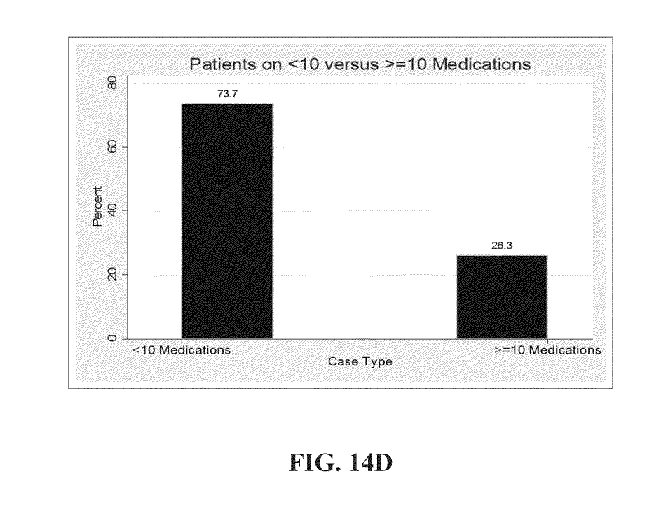

FIGS. 14A-D illustrate the data for 5311 patients in the trial conducted. FIGS. 14A-B illustrate the number of patients at each age group and the number of medications that the patients are on. FIGS. 14C-D illustrate the incidence of polypharmacy in the test patient population.

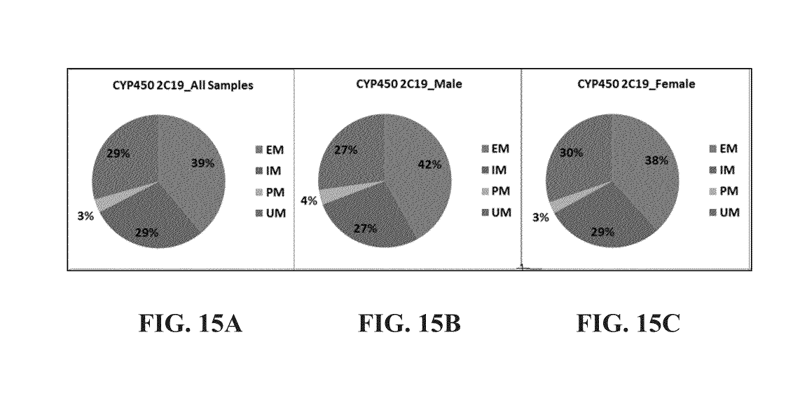

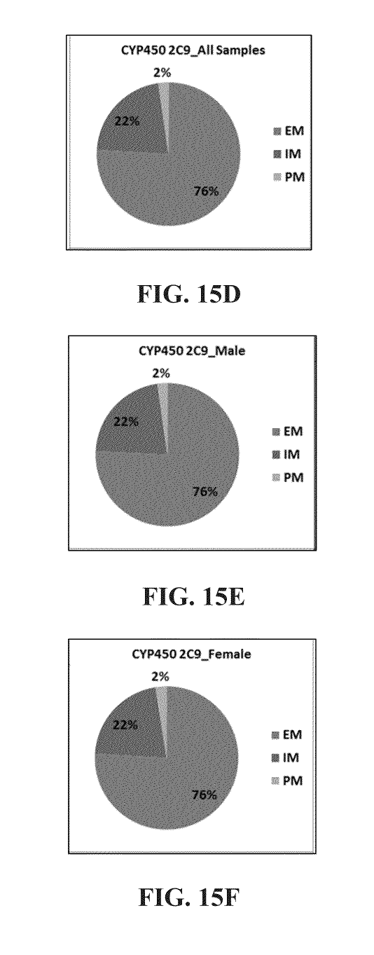

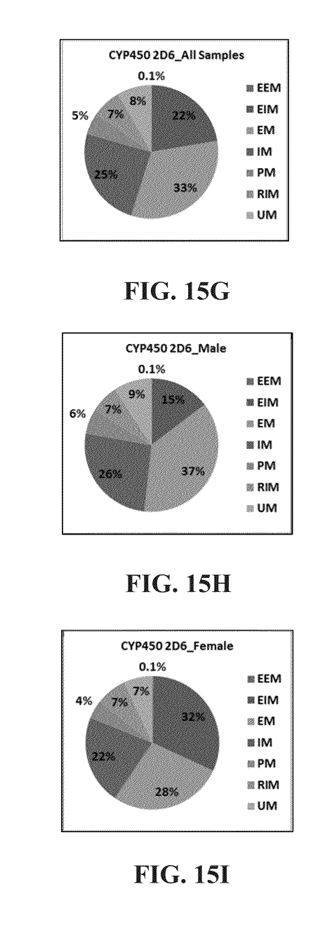

Pharmacogenomics tests were conducted on the FLEX trial patients, in accordance with several embodiments disclosed herein. The results of the tests are shown in FIGS. 15A-I which illustrate the individual CYP2C9, CYP2C19 and CYP2D6 phenotype frequencies and the patient's level of metabolism for each: “UM”—ultrarapid metabolizer; “EM”—extensive metabolizer; “RIM”—reduced intermediate metabolizer; “EIM”—enhanced intermediate metabolizer; “TM”—intermediate metabolizer”; “PM”—poor metabolizer; or “EEM”—enhanced extensive metabolizer.

FIGS. 15A-C illustrate the distribution of different genotypes of patient metabolism in all patients for CYP2C19, all males for CYP2C19, and all females for CYP2C19. For all patients sampled, 3% had a poor metabolizer genotype; 29% had an intermediate metabolizer genotype, 29% had an ultrarapid metabolizer genotype, and 39% had an enhanced intermediate metabolizer genotype. For all males sampled, 4% had a poor metabolizer genotype; 27% had an intermediate metabolizer genotype, 27% had an ultrarapid metabolizer genotype, and 42% had an enhanced intermediate metabolizer genotype. For all females sampled, 3% had a poor metabolizer genotype; 29% had an intermediate metabolizer genotype, 30% had an ultrarapid metabolizer genotype, and 38% had an enhanced intermediate metabolizer genotype.

FIGS. 15D-F illustrate the distribution of different genotypes of patient metabolism in all patients for CYP2C9, all males for CYP2C9, and all females for CYP2C9. For all patients sampled, 2% had a poor metabolizer genotype; 22% had an intermediate metabolizer genotype, and 76% had an enhanced intermediate metabolizer genotype. For all males sampled, 2% had a poor metabolizer genotype; 22% had an intermediate metabolizer genotype, and 76% had an enhanced intermediate metabolizer genotype. For all females sampled, 2% had a poor metabolizer genotype; 22% had an intermediate metabolizer genotype, and 76% had an enhanced intermediate metabolizer genotype.

FIGS. 15G-I illustrate the distribution of different genotypes of patient metabolism in all patients for CYP2D6, all males for CYP2D6, and all females for CYP2D6. For all patients sampled, 0.1% had an enhanced extensive metabolizer genotype, 22% had an enhanced intermediate metabolizer genotype, 33% had an extensive metabolizer genotype, 25% had an intermediate metabolizer genotype, 5% had a poor metabolizer genotype, 7% had a reduced intermediate metabolizer genotype, and 8% had an ultrarapid metabolizer genotype. For all males sampled, 0.1% had an enhanced extensive metabolizer genotype, 15% had an enhanced intermediate metabolizer genotype, 37% had an extensive metabolizer genotype, 26% had an intermediate metabolizer genotype, 6% had a poor metabolizer genotype, 7% had a reduced intermediate metabolizer genotype, and 9% had an ultrarapid metabolizer genotype. For all females sampled, 0.1% had an enhanced extensive metabolizer genotype, 32% had an enhanced intermediate metabolizer genotype, 28% had an extensive metabolizer genotype, 22% had an intermediate metabolizer genotype, 4% had a poor metabolizer genotype, 7% had a reduced intermediate metabolizer genotype, and 7% had an ultrarapid metabolizer genotype. The results of the pharmacogenomics test supported the phenotypic distribution of metabolizers in the patient population.

### Results: Significant Clinical Findings Supporting Pharmacogenomics.

The statistical findings from the FLEX trial found strong associations between age groups and the quantity of medications taken. Up until the ages of 25-39, only 9.36% of patients within a group were taking 10 or more medications. For age groups 40-54 and 55-64, the percentage of patients taking 10 or more medications increased to 21.75% and 35.02% respectively. The FLEX trial also revealed that elderly patients are at a greater risk of adverse reactions or lack of efficacy due to the increase in quantity of medications taken.

The statistical findings from the FLEX trial also studied the percent of the test patient population that had normal metabolizers for CYP2D6, CYP2C19, CYP2C9, CYP3A4 and CYP3A5. 14.1% of 5,311 patients were found to have extensive (normal) metabolism for CYP2D6 AND CYP2C19. 8.5% of 5,311 patients were found to have extensive (normal) metabolism for CYP2D6, CYP2C19, and CYP2C9. Finally, only 25 of 5,311 (0.5%) of patients had extensive (normal) metabolism) for all CYP450 genes that were considered. Of those 25 individuals, 19 of them were taking less than 10 medications whereas only 6 of them were on over 10 medications. Therefore, these study results suggest that 99.5% of patients overall have at least one genetic variant amongst the CYP450 genes. As such, these experiments reinforce the importance of affirmative genomic analysis of cytochrome P450 enzyme expression and/or activity after a threshold analysis has been performed (in some embodiments) in order to determine whether a particular patients genomic profile renders them eligible for possible clozapine administration. As can be appreciated from the high-level of genetic variants among the cytochrome P450 enzymes, even after passing the threshold determination to become eligible for clozapine administration, a patient's from good genomic analysis may eventually country indicate use of clozapine, because of, for example, an extremely high or an extremely low metabolism of the drug. The former could make achieving therapeutic levels of the drug in the patient's body a challenge and could increase the risk of side effects because of the dose is required to overcome an ultra-rapid metabolism of the drug. The latter could likewise present an increased risk of adverse side effects because a given dose of the drug may remain in a poor metabolizers system for an extended period of time, even if at low concentrations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein.

When the singular forms “a,” “an” and “the” or like terms are used herein, they will be understood to include plural referents unless the context clearly dictates otherwise. Thus, for example, reference to “an agent” includes two or more agents, and the like. The word “or” or like terms as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering clozapine” include “instructing the administration of clozapine.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 3 mm” includes “3 mm.”

What is claimed is:

1. A method for treating a schizophrenic patient with an individualized dosing regimen, the method comprising:
identifying a schizophrenic patient resistant to at least one antipsychotic medication and under consideration for receiving clozapine;
ordering at least one genomic test, the at least one genomic test comprising;
(i) screening genetic material of patient for a sequence variant in at least one gene associated with increased risk of clozapine-induced agranulocytosis,
wherein the sequence variants are present in a gene selected from the group consisting of HLA-DQB1, HLA-C, DRD1, NTSR1 and CSF2RB;
(ii) determining, when said screening identifies at least one sequence variant, the expression level of one or more enzymes capable of metabolizing clozapine;
(iii) categorizing the patient's capacity to metabolize clozapine based on the determined expression of the one or more enzymes capable of metabolizing clozapine; and
(iv) calculating an individualized clozapine dosing regimen for the patient based on the categorization of the patient's capacity to metabolize clozapine; and
treating the patient with the individualized clozapine dosing regimen.

2. The method of claim 1, wherein said screening further comprises obtaining genetic material from a biological sample from the patient.

3. The method of claim 2, wherein the at least one gene associated with increased risk of clozapine-induced agranulocytosis comprises HLA-DQB1.

4. The method of claim 3, wherein the sequence variant comprises a 6672G>C substitution in HLA-DQB1.

5. The method of claim 1, wherein the calculation of the individualized clozapine dosing regimen further comprises an analysis of one or more lifestyle factors or other diseases that may impact the metabolism of clozapine.

6. The method of claim 1, wherein the one or more enzymes capable of metabolizing clozapine comprises at least one cytochrome p450 oxidase (CYP450).

7. The method of claim 6, wherein the CYP450 is selected from the group consisting of CYP450 2D6, CYP450 1A2, and CYP450 3A4.

8. The method of claim 1, wherein categorizing the subject's expression of the enzyme further comprises determining the metabolic status for the one or more enzymes capable of metabolizing clozapine.

9. The method of claim 8, wherein the metabolic status determined for each of the enzymes is that of ultra-rapid metabolizer, extensive metabolizer, intermediate metabolizer, and poor metabolizer.

10. The method of claim 1, wherein the method further comprises ordering an analysis of putative drug-drug interactions that may alter the metabolism of clozapine, and wherein the calculating of the individualized clozapine dosing regimen is adjusted to account for any negative drug-drug interactions that increase or decrease the metabolism of clozapine.

11. The method of claim 1, wherein the screening is performed by a method selected from the group consisting of next generation sequencing (NGS), real-time RT-PCR, micro-array analysis.

12. The method of claim 1, wherein the determining is performed by a method selected from the group consisting of next generation sequencing (NGS), real-time RT-PCR, micro-array analysis.

13. The method of claim 1, wherein the genetic material comprises saliva obtained from a cheek swab of the patient or blood drawn from the patient.

14. A method for treating a schizophrenic patient with an individualized clozapine dosing regimen, the method comprising:
identifying a schizophrenic patient under consideration for receiving clozapine;
ordering a test comprising;
(i) screening genetic material isolated from a biological sample collected from the patient for the presence of a 6672G>C substitution in a HLA-DQB1 gene using next generation sequencing (NGS), wherein said substitution is associated with increased risk of clozapine-induced agranulocytosis,
(ii) determining, when said NGS screening identifies said substitution, the expression level each of cytochrome p450 oxidase (CYP450) 1A2, CYP450 2D6, and CYP450 3A4;
(iii) categorizing the patient's capacity to metabolize clozapine as that of ultra-rapid metabolizer, extensive metabolizer, intermediate metabolizer, or poor metabolizer for each of CYP450 1A2, CYP450 2D6, and CYP450 3A4 based on the determined expression each of CYP450 1A2, CYP450 2D6, and CYP450 3A4;
(iv) determining the patient's overall capacity to metabolize clozapine based on the categorization;
(v) adjusting a starting dose of clozapine based on the categorization of the determined capacity of the patient to metabolize clozapine, thereby generating an individualized clozapine dosing regimen for the patient; and
treating the patient with the individualized clozapine dosing regimen.

15. The method of claim 14, further comprising obtaining at least one blood sample after treating said patient, and evaluating the white blood cell profile of the sample to determine if the patient is developing agranulocytosis.

16. The method of claim 15, further comprising adjusting the individualized clozapine dosing regimen to maintain a clinically acceptable white blood cell profile.

17. A method for calculating an individualized clozapine dosing regimen, the method comprising:
receiving a biological sample collected from a schizophrenic patient under consideration for receiving clozapine;
screening genetic material isolated from the biological sample for a sequence variant in at least one gene associated with increased risk of clozapine-induced agranulocytosis;
determining, when said screening identifies said sequence variant, the expression level of at least one cytochrome p450 oxidase (CYP450) enzyme;
categorizing the patient's capacity to metabolize clozapine as that of ultra-rapid metabolizer, extensive metabolizer, intermediate metabolizer, or poor metabolizer for each CYP450 for which expression is determined;
determining the patient's overall capacity to metabolize clozapine based on the categorization;
obtaining a starting dose of clozapine based on a clinically acceptable use of clozapine;
adjusting the starting dose of clozapine based on the categorization of the determined capacity of the patient to metabolize clozapine, thereby calculating an individualized clozapine dosing regimen for the patient.

18. The method of claim 17, wherein the sequence variant comprises a 6672G>C substitution in a HLA-DQB1 gene using next generation sequencing (NGS); wherein said determining the expression level of at least one CYP450 comprises determining the expression of CYP450 1A2, CYP450 2D6, and CYP450 3A4.

19. The method of claim 17, further comprising increasing or decreasing the dose of the clozapine when the patient is also receiving one or more drugs that alters the patient's capacity to metabolize clozapine and/or when the patient has one or more lifestyle characteristics that alters the patient's capacity to metabolize clozapine.

20. The method of claim 17, further comprising reporting said calculated individualized clozapine dosing regimen to a medical care provider for administration to the patient.
